(19) United States
(12) Patent Application Publication
(10) Pub. No.: US 2025/0026833 A1
(43) Pub. Date: Jan. 23, 2025

(54) A BISPECIFIC ANTI-PD-L1/VEGF ANTIBODY AND USES THEREOF

(71) Applicant: WUXI BIOLOGICS IRELAND LIMITED, Louth (IE)

(72) Inventors: Yi QIN, Shanghai (CN); Zhuozhi WANG, Shanghai (CN); Yunying CHEN, Shanghai (CN); Jing LI, Shanghai (CN); Jijie GU, Shanghai (CN)

(21) Appl. No.: 18/282,767

(22) PCT Filed: Mar. 30, 2022
(86) PCT No.: PCT/CN2022/084086

§ 371 Date: Sep. 18, 2023

(30) Foreign Application Priority Data
Mar. 31, 2021 (WO) PCT/CN2021/084447

(51) Int. Cl. C07K 16/28; A61K 39/00; A61K 39/395; A61K 45/06; A61P 35/00; C07K 16/22

(52) U.S. Cl. CPC C07K 16/2827; A61K 39/3955; A61K 45/06; A61P 35/00; C07K 16/22; A61K 2039/505; C07K 2317/31; C07K 2317/62

(57) ABSTRACT

The present disclosure provides bispecific anti-VEGF×PD-L1 antibody or antigen-binding portion thereof, methods of producing the bispecific antibody or antigen-binding portion thereof, methods of treating diseases or conditions using the bispecific antibody or antigen-binding portion thereof.

### CROSS REFERENCE

This application is a § 371 national phase of International Application No. PCT/CN2022/084086, filed on Mar. 30, 2022, which claims the benefit of International application PCT/CN2021/084447, filed on Mar. 31, 2021, which applications are incorporated by reference in their entireties.

### SEQUENCE LISTING

The present application is filed with a Sequence Listing in electronic form. The entire contents of the Sequence Listing are hereby incorporated by reference.

### FIELD

The present disclosure generally relates to bispecific anti-PD-L1×VEGF antibodies, a method for preparing the same and uses thereof.

### BACKGROUND

Angiogenesis is essential for the growth of tumor and the development of metastasis. Controlling tumor-associated angiogenesis is a promising strategy for cancer therapy. Vascular endothelial growth factor (VEGF) is the key mediator of angiogenesis and has been validated in various types of human cancers [1]. Tumor cells release growth factors such as VEGF that bind to nearby endothelial cells, which initiates a signaling cascade that stimulates endothelial cells to divide and form new blood vessels. VEGF signaling through its receptors VEGFR plays a critical role in angiogenesis and growth of many solid tumors. Antiangiogenic drugs, such as Avastin (Bevacizumab) which targets the VEGF pathway, have achieved a success in clinic.

On the other hand, targeting immune checkpoint molecules, such as programmed death ligand 1 (PD-L1) or its receptor, programmed death 1 (PD-1), has shown promising clinical success [2, 3]. PD-L1 expression strongly correlates with unfavorable prognosis in various types of cancers. Anti-PD-L1 antibody can target PD-L1 expressed on tumor cells and tumor-infiltrating immune cells, and prevent binding to PD-1 and B7.1 on the surface of T cells, and also enable the activation of T cells as well as recruit other T cells to attack the tumor, then empower the immune system to fight multiple types of cancer.

Anti-VEGF therapy, in addition to its established anti-angiogenic effects, may further enhance anti-PD-1/PD-L1 therapy's ability to restore anti-cancer immunity, by inhibiting VEGF-related immunosuppression, promoting T-cell tumor infiltration and enabling priming and activation of T-cell responses against tumor antigens [4, 5]. Therefore, developing VEGF and PD-L1 bispecific antibodies which combine anti-angiogenesis therapy and immune checkpoint inhibition together could achieve promising results in cancer therapy.

Despite the obvious benefit of targeting VEGF and also targeting PD-1/PD-L1 therapies, there is still a significant unmet need exist. 15%-20% patients do not response to anti-VEGF treatment, and increasing evidence has indicated that prolonged use of anti-VEGF agents for cancer therapy promotes tumor resistance. 3%-9% patients develop immunogenicity of the treatment. And also the limited overall survival time extension and limited safety issue, including changes in bone morphology, glomerulopathy with inflammation in kidney, and decreased vacuolation with inflammation in adrenal gland. Immune checkpoint inhibitors blocking the PD-1/PD-L1 pathway, such as nivolumab, pembrolizumab and atezolizumab, represent a standard treatment option for patients with multiple cancer. However, with a response rate of 14-23% in unselected populations and 16-48% in patients with PD-L1-expressing tumors, these drugs offer improved outcomes in some patients, but not all.

Therefore, there is great need to develop novel anti-PD-L1/anti-VEGF bispecific antibodies. In the present disclosure, a bispecific antibody that could simultaneously bind to human PD-L1 and VEGF with high affinity, block both PD-1/PD-L1 and VEGF/VEGFR signaling, and display superior anti-tumor efficacy has been generated.

### SUMMARY

These and other objectives are provided for by the present disclosure which, in a broad sense, is directed to compounds, methods, compositions and articles of manufacture that provide antibodies with improved efficacy. The benefits provided by the present disclosure are broadly applicable in the field of antibody therapeutics and diagnostics and may be used in conjunction with antibodies that react with a variety of targets.

In one aspect, the present disclosure provides a bispecific antibody or antigen-binding portion thereof, comprising a PD-L1 antigen-binding moiety and a VEGF antigen-binding moiety.

In some embodiments, the present disclosure provides a bispecific antibody or antigen-binding portion thereof, comprising a PD-L1 antigen-binding moiety associated with a VEGF antigen-binding moiety, wherein:
the PD-L1 antigen-binding moiety comprises: a heavy chain complementarity determining region (HCDR) 1 comprising the amino acid sequence of SEQ ID NO: 1, a HCDR2 comprising the amino acid sequence of SEQ ID NO: 2, a HCDR3 comprising the amino acid sequence of SEQ ID NO: 3, a light chain complementarity determining region (LCDR) 1 comprising the amino acid sequence of SEQ ID NO: 4, a LCDR2 comprising the amino acid sequence of SEQ ID NO: 5, and a LCDR3 comprising the amino acid sequence of SEQ ID NO: 6; and
the VEGF antigen-binding moiety comprises: a HCDR1 comprising the amino acid sequence of SEQ ID NO: 7, a HCDR2 comprising the amino acid sequence of SEQ ID NO: 8, a HCDR3 comprising the amino acid sequence of SEQ ID NO: 9, a LCDR1 comprising the amino acid sequence of SEQ ID NO: 10, a LCDR2 comprising the amino acid sequence of SEQ ID NO: 11, and a LCDR3 comprising the amino acid sequence of SEQ ID NO: 12.

In certain embodiements, the PD-L1 antigen-binding moiety is a scFv and the VEGF antigen-binding moiety is a Fab.

In certain embodiements, the PD-L1 antigen-binding moiety comprises a heavy chain variable domain comprising the amino acid sequence of SEQ ID NO: 13 or an amino acid sequence with at least 85%, 90%, or 95% identity to SEQ ID NO: 13 and a light chain variable domain comprising the amino acid sequence of SEQ ID NO:14 or an amino acid sequence with at least 85%, 90%, or 95% identity to SEQ ID NO: 14.

In certain embodiments, the VEGF antigen-binding moiety comprises a heavy chain variable domain comprising the amino acid sequence of SEQ ID NO: 15 or an amino acid sequence with at least 85%, 90%, or 95% identity to SEQ ID NO: 15 and a light chain variable domain comprising the amino acid sequence of SEQ ID NO:16 or an amino acid sequence with at least 85%, 90%, or 95% identity to SEQ ID NO: 16.

In certain embodiements, the PD-L1 antigen-binding moiety is fused to the N terminal of the VEGF antigen-binding moiety. In some other embodiements, the PD-L1 antigen-binding moiety is fused to the C terminal of the VEGF antigen-binding moiety.

In certain embodiements, the PD-L1 antigen-binding moiety is operably linked to the N terminal of the light chain or heavy chain of the VEGF antigen-binding moiety, optionally via a linker. The linker may comprise or consist of 1 to 4 copies of GGGGS (G4S), for example, the linker may be (G4S)₂.

In certain embodiements, the bispecific antibody or antigen-binding portion thereof as disclosed herein comprises a heavy chain and a light chain, wherein:
the heavy chain comprises, from N-terminal to C-terminal, domains operably linked as in scFv-VH-CH1-hinge-Fc, wherein the scFv is from the PD-L1 antigen-binding moiety and the VH-CH1 is from the VEGF antigen-binding moiety; and
the light chain comprises, from N-terminal to C-terminal, domains operably linked as in VL-CL, wherein the VL-CL is from the VEGF antigen-binding moiety.

In certain embodiements, the Fc region is a human IgG Fc region, preferably a human IgG1 Fc region or a variant thereof. Specifically, the Fc region of the bispecific antibody comprises L234A and L235A substitutions, according to EU numbering.

In certain embodiements, the bispecific antibody or antigen-binding portion thereof as disclosed herein comprise a heavy chain comprising SEQ ID NO: 17 and a light chain comprising SEQ ID NO: 18.

In certain embodiements, the bispecific antibody or antigen-binding portion thereof as disclosed herein is a humanized antibody.

In one aspect, the present disclosure provides an isolated nucleic acid molecule, comprising a nucleic acid sequence encoding the bispecific antibody or the antigen-binding portion thereof as disclosed herein.

In one aspect, the present disclosure provides a vector comprising the nucleic acid molecule as disclosed herein. In one aspect, the present disclosure provides a host cell comprising the nucleic acid molecule or the vector as disclosed herein.

In one aspect, the present disclosure provides a pharmaceutical composition comprising the bispecific antibody or the antigen-binding portion thereof as disclosed herein and a pharmaceutically acceptable carrier.

In one aspect, the present disclosure provides a method for producing the bispecific antibody or the antigen-binding portion thereof as disclosed herein, comprising the steps of:
expressing the bispecific antibody or the antigen-binding portion thereof in a host cell comprising a nucleic acid molecule(s) or vector(s) encoding the bispecific antibody or the antigen-binding portion thereof; and
isolating the bispecific antibody or antigen-binding portion thereof from the host cell.

In one aspect, the present disclosure provides a method for modulating an immune response in a subject, comprising administering to the subject the bispecific antibody or the antigen-binding portion thereof or the pharmaceutical composition as disclosed herein to the subject, optionally the immune response is PD-L1 and/or VEGF related.

In one aspect, the present disclosure provides a method for inhibiting growth of tumor cells in a subject, comprising administering an effective amount of the bispecific antibody or the antigen-binding portion thereof or the pharmaceutical composition as disclosed herein to the subject.

In one aspect, the present disclosure provides a method for preventing or treating cancer in a subject, comprising administering an effective amount of the bispecific antibody or the antigen-binding portion thereof or the pharmaceutical composition to the subject. The cancer may be PD-L1 and/or VEGF related. In certain embodiements, the cancer to be treated is colon cancer or colorectal cancer.

In certain embodiements, the bispecific antibody or antigen-binding portion thereof as disclosed herein may be administered in combination with a chemotherapeutic agent, radiation and/or other agents for use in cancer immunotherapy.

In one aspect, the present disclosure provides a bispecific antibody or antigen-binding portion thereof as disclosed herein for use
i) in the modulation of PD-L1 and/or VEGF related immune responses;
ii) in enhancing T cell proliferation and cytokine production; and/or
iii) in stimulating an immune response or function, such as boosting the immune response against cancer cells.

In one aspect, the present disclosure provides the bispecific antibody or antigen-binding portion thereof as disclosed herein for use in diagnosing, preventing or treating cancers.

In one aspect, the present disclosure provides use of the bispecific antibody or antigen-binding portion thereof as disclosed herein in the manufacture of a medicament for modulating an immune response or inhibiting growth of tumor cells in a subject.

In one aspect, the present disclosure provides use of the bispecific antibody or antigen-binding portion thereof as disclosed herein in the manufacture of a medicament for diagnosing, treating or preventing cancers.

In one aspect, the present disclosure provides a kit comprising the bispecific antibody or antigen-binding portion thereof as disclosed herein. The kit can be used for detection, diagnosis, prognosis, or treatment of a disease or condition, such as cancer.

The foregoing is a summary and thus contains, by necessity, simplifications, generalizations, and omissions of detail; consequently, those skilled in the art will appreciate that the summary is illustrative only and is not intended to be in any way limiting. Other aspects, features, and advantages of the methods, compositions and/or devices and/or other subject matter described herein will become apparent in the teachings set forth herein. The summary is provided to introduce a selection of concepts in a simplified form that are further described below in the Detailed Description. This summary is not intended to identify key features or essential features of the claimed subject matter, nor is it intended to be used as an aid in determining the scope of the claimed subject matter. Further, the contents of all references, patents and published patent applications cited throughout this application are incorporated herein in entirety by reference.

### BRIEF DESCRIPTION OF FIGURES

FIG. 1 shows a schematic representation of the format of W3253 antibody.

FIG. 2 shows SDS-PAGE of W3253-U9T2.G17-1.uIgG1V320 (lane 1). 1: Non-reducing status, 1′: reducing status in NuPAGE (Novex 4-12% Bis-Tris) gel. M, PageRuler™ Unstained Protein Ladder.

FIG. 3 shows the HPLC-SEC result of W3253-U9T2.G17-1.uIgG1V320.

FIG. 4 shows the ELISA binding result of W3253-U9T2.G17-1.uIgG1V320 to human VEGF (same as cyno VEGF).

FIG. 5 shows the FACS binding result of W3253-U9T2.G17-1.uIgG1V320 to human PD-L1.

FIG. 6 shows the dual binding result of W3253-U9T2.G17-1.uIgG1V320 to VEGF and then PD-L1.

FIG. 7 shows the FACS binding result of W3253-U9T2.G17-1.uIgG1V320 to cynomolgus PD-L1.

FIGS. 8A, 8B and 9 show the SPR sensorgram of W3253-U9T2.G17-1.uIgG1V320 binding to human PD-L1 (FIG. 8A), cynomolgus PD-L1 (FIG. 8B) and human VEGF (FIG. 9).

FIGS. 10-12 show the competition of W3253-U9T2.G17-1.uIgG1V320 to human VEGFR1 (FIG. 10) and VEGFR2 (FIG. 11) on human VEGF binding, and the competition of W3253-U9T2.G17-1.uIgG1V320 to human PD-1 on human PD-L1 binding (FIG. 12).

FIG. 13 shows the inhibition by antibodies on HUVEC cells proliferation.

FIG. 14 shows the effect of antibodies on PD-L1 reporter gene assay.

FIGS. 15A-15B shows the effect of antibodies on hCD4+T cell IL-2 (FIG. 15A) and IFN-γ (FIG. 15B) secretion in MLR assay.

FIG. 16 shows the effect of antibodies in human serum stability test by dual binding ELISA.

FIG. 17 shows the DSF profiles of W3253-U9T2.G17-1.uIgG1V320.

FIG. 18 shows pharmacokinetics profiles of W3253-U9T2.G17-1.uIgG1V320 in mouse detected by different methods.

FIGS. 19-20 show body weight (FIG. 19) and tumor volume (FIG. 20) of each group in mixed RKO-PBMC model after treatment by different antibodies.

FIGS. 21-22 show body weight (FIG. 21) and tumor volume (FIG. 22) of each group in human PD-L1 knock-in MC38+human PD1/PD-L1 Dual knock-in transgenic mouse model after treatment by different antibodies.

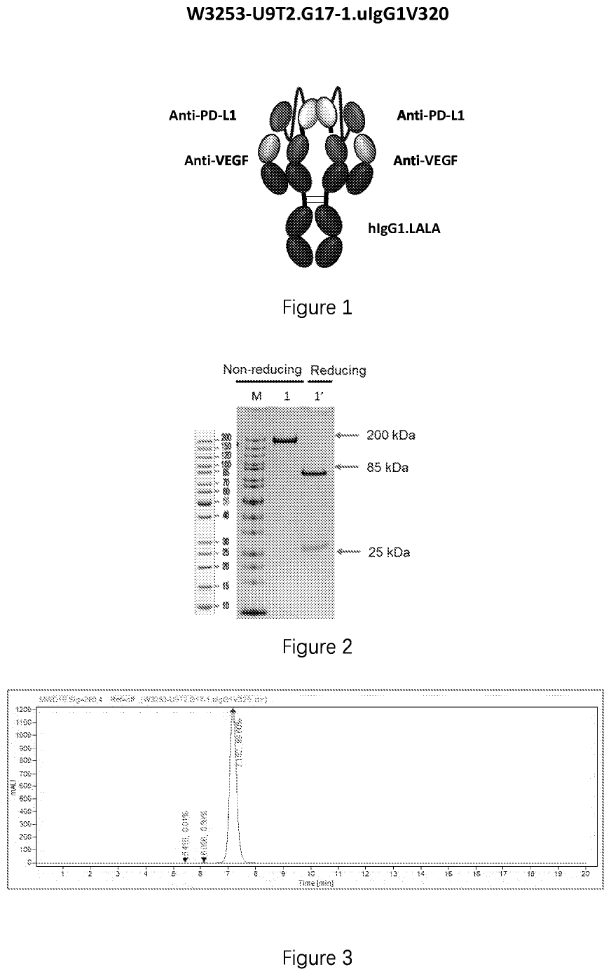

### DETAILED DESCRIPTION

While the present disclosure may be embodied in many different forms, disclosed herein are specific illustrative embodiments thereof that exemplify the principles of the disclosure. It should be emphasized that the present disclosure is not limited to the specific embodiments illustrated. Moreover, any section headings used herein are for organizational purposes only and are not to be construed as limiting the subject matter described.

Unless otherwise defined herein, scientific and technical terms used in connection with the present disclosure shall have the meanings that are commonly understood by those of ordinary skill in the art. Further, unless otherwise required by context, singular terms shall include pluralities and plural terms shall include the singular. More specifically, as used in this specification and the appended claims, the singular forms “a,” “an” and “the” include plural referents unless the context clearly dictates otherwise. Thus, for example, reference to “a protein” includes a plurality of proteins; reference to “a cell” includes mixtures of cells, and the like. In this application, the use of “or” means “and/or” unless stated otherwise. Furthermore, the use of the term “comprising,” as well as other forms, such as “comprises” and “comprised,” is not limiting. In addition, ranges provided in the specification and appended claims include both end points and all points between the end points.

Generally, nomenclature used in connection with, and techniques of, cell and tissue culture, molecular biology, immunology, microbiology, genetics and protein and nucleic acid chemistry and hybridization described herein are those well-known and commonly used in the art. The methods and techniques of the present disclosure are generally performed according to conventional methods well known in the art and as described in various general and more specific references that are cited and discussed throughout the present specification unless otherwise indicated. See, e.g., Abbas et al., Cellular and Molecular Immunology, 6^{th} ed., W.B. Saunders Company (2010); Sambrook J. & Russell D. Molecular Cloning: A Laboratory Manual, 3rd ed., Cold Spring Harbor Laboratory Press, Cold Spring Harbor, N.Y. (2000); Ausubel et al., Short Protocols in Molecular Biology: A Compendium of Methods from Current Protocols in Molecular Biology, Wiley, John & Sons, Inc. (2002); Harlow and Lane Using Antibodies: A Laboratory Manual, Cold Spring Harbor Laboratory Press, Cold Spring Harbor, N.Y. (1998); and Coligan et al., Short Protocols in Protein Science, Wiley, John & Sons, Inc. (2003). The nomenclature used in connection with, and the laboratory procedures and techniques of, analytical chemistry, synthetic organic chemistry, and medicinal and pharmaceutical chemistry described herein are those well-known and commonly used in the art.

### Definitions

In order to better understand the disclosure, the definitions and explanations of the relevant terms are provided as follows.

The term “antibody” or “Ab,” herein is used in the broadest sense, which encompasses various antibody structures, including polyclonal antibodies, monospecific and multispecific antibodies (e.g. bispecific antibodies). A native intact antibody generally is a Y-shaped tetrameric protein comprising two heavy (H) and two light (L) polypeptide chains held together by covalent disulfide bonds and non-covalent interactions. Light chains of an antibody may be classified into κ and λ light chain. Heavy chains may be classified into μ, δ, γ, α and ε, which define isotypes of an antibody as IgM, IgD, IgG, IgA and IgE, respectively. In a light chain and a heavy chain, a variable region is linked to a constant region via a “J” region of about 12 or more amino acids, and a heavy chain further comprises a “D” region of about 3 or more amino acids. Each heavy chain consists of a heavy chain variable region (VH) and a heavy chain constant region (CH). A heavy chain constant region consists of 3 domains (CH1, CH2 and CH3). Each light chain consists of a light chain variable region (VL) and a light chain constant region (CL). VH and VL region can further be divided into hypervariable regions (called complementary determining regions (CDR)), which are interspaced by relatively conservative regions (called framework region (FR)). Each VH and VL consists of 3 CDRs and 4 FRs in the following order: FR1, CDR1, FR2, CDR2, FR3, CDR3, FR4 from N-terminal to C-terminal. The variable region (V_{H} and V_{L}) of each heavy/light chain pair forms antigen binding sites, respectively. Distribution of amino acids in various regions or domains follows the definition in Kabat Sequences of Proteins of Immunological Interest (National Institutes of Health, Bethesda, Md. (1987 and 1991)), or Chothia & Lesk (1987) J. Mol. Biol. 196:901-917; Chothia et al., (1989) Nature 342:878-883. Antibodies may be of different antibody isotypes, for example, IgG (e.g., IgG1, IgG2, IgG3 or IgG4 subtype), IgA1, IgA2, IgD, IgE or IgM antibody.

The term “antigen-binding portion” or “antigen-binding fragment” of an antibody, which can be interchangeably used in the context of the application, refers to polypeptides comprising fragments of a full-length antibody, which retain the ability of specifically binding to an antigen that the full-length antibody specifically binds to, and/or compete with the full-length antibody for binding to the same antigen. Generally, see Fundamental Immunology, Ch. 7 (Paul, W., ed., the second edition, Raven Press, N.Y. (1989), which is incorporated herein by reference for all purposes. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein. In certain embodiments, an antigen-binding fragment of an antibody may contain at least one variable domain covalently linked to at least one constant domain. The variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule.

The term “variable domain” with respect to an antibody as used herein refers to an antibody variable region or a fragment thereof comprising one or more CDRs. Although a variable domain may comprise an intact variable region (such as HCVR or LCVR), it is also possible to comprise less than an intact variable region yet still retain the capability of binding to an antigen or forming an antigen-binding site.

The term “antigen-binding moiety” as used herein refers to an antibody fragment formed from a portion of an antibody comprising one or more CDRs, or any other antibody fragment that binds to an antigen but does not comprise an intact native antibody structure. Different from the term “antigen binding site” which generally refers to the variable domains, an antigen-binding moiety may comprise constant domains in addition to variable domains. Examples of antigen-binding moiety include, without limitation, a variable domain, a variable region, a diabody, a Fab, a Fab′, a F(ab′)₂, an Fv fragment, a disulfide stabilized Fv fragment (dsFv), a (dsFv)₂, a bispecific dsFv (dsFv-dsFv′), a disulfide stabilized diabody (ds diabody), a multispecific antibody, a camelized single domain antibody, a nanobody, a domain antibody, and a bivalent domain antibody. An antigen-binding moiety is capable of binding to the same antigen to which the parent antibody binds. In certain embodiments, an antigen-binding moiety may be a Fab fragment or a VHH antibody. In some embodiments, an antigen-binding moiety may comprise one or more CDRs from a particular human antibody grafted to a framework region from one or more different human antibodies. For more and detailed formats of antigen-binding moiety are described in Spiess et al., Molecular Immunology, 67(2), pp. 95-106 (2015), and Brinkman et al., mAbs, 9(2), pp. 182-212 (2017), which are incorporated herein by their entirety.

“Fab” with regard to an antibody refers to that portion of the antibody consisting of a single light chain (both variable and constant regions) associating to the variable region and first constant region of a single heavy chain by a disulfide bond.

The term “ScFv” or “single-chain fragment variable”, as used herein, refers to a fusion protein of the variable regions of the heavy and light chains of immunoglobulins (VH and VL), generally connected by a short linker peptide (e.g. about 6 to 25 amino acids in length). It has been found that the small size of scFvs allows for higher loading capacities and superior orientation of targeting ligands, leading to overall improvements in efficacy.

“Fc” (short for fragment, crystallizable) with regard to an antibody refers to that portion of the antibody comprising the second (CH2) and third (CH3) constant regions of a first heavy chain bound to the second and third constant regions of a second heavy chain via disulfide bonding. When referring to a Fc region, depending on the context, it may refer to one chain or both chains of the Fc region. The Fc portion of the antibody is responsible for various effector functions such as ADCC, and CDC, but does not function in antigen binding. The capacity of antibodies to initiate and regulate effector functions through their Fc domain is a key component of their in vivo protective activity. Although the neutralizing activity of antibodies has been previously considered to be solely the outcome of Fab-antigen interactions, it has become apparent that their in vivo activity is highly dependent on interactions of the IgG Fc domain with its cognate receptors, Fcγ receptors (FcγRs), expressed on the surface of effector leukocytes.

The term “PD-L1”, also known as programmed death-ligand 1, is a 40 kDa type 1 transmembrane protein that has been speculated to play a major role in suppressing the adaptive arm of immune system. PD-L1 is the principal ligand of programmed death 1 (PD-1), a coinhibitory receptor that can be constitutively expressed or induced in myeloid, lymphoid, normal epithelial cells and in cancer. The term “PD-L1” as used herein, when referring to the amino acid sequence of PD-L1 protein, including full-length PD-L1 protein, or the extracellular domain of PD-L1 (PD-L1 ECD) or fragment containing PD-L1 ECD; Fusion protein of PD-L1 ECD, for example, fragment fused with IgG Fc from mice or human (mFc or hFc) is also included. Moreover, as understood by a person skilled in the art, PD-L1 protein would also include those into which mutations of amino acid sequence are naturally or artificially introduced (including but not limited to replacement, deletion and/or addition) without affecting the biological functions.

The term “an antibody that binds PD-L1” or an “anti-PD-L1 antibody” as used herein includes antibodies and antigen-binding fragments thereof that specifically recognize PD-L1. The antibodies and antigen-binding fragments of the present disclosure may bind soluble PD-L1 protein and/or cell surface expressed PD-L1. Soluble PD-L1 includes natural PD-L1 proteins as well as recombinant PD-L1 protein variants that lack a transmembrane domain or are otherwise unassociated with a cell membrane. As used herein, the expression “anti-PD-L1 antibody” includes both monovalent antibodies with a single specificity, as well as bispecific antibodies comprising a first antigen-binding site that binds PD-L1 and a second antigen-binding site that binds a second (target) antigen, wherein the anti-PD-L1 antigen-binding site comprises any of the HCVR/LCVR or CDR sequences as set forth in Table A herein. Examples of anti-PD-L1 bispecific antibodies are described elsewhere herein. The term “antigen-binding molecule” includes antibodies and antigen-binding fragments of antibodies, including, e.g., bispecific antibodies.

The term “VEGF” (vascular endothelial growth factor, also known as VEGF-A), is a signal protein produced by cells that stimulates the formation of blood vessels. VEGF is a sub-family of growth factors, the platelet-derived growth factor family of cystine-knot growth factors. They are important signaling proteins involved in both vasculogenesis (the de novo formation of the embryonic circulatory system) and angiogenesis (the growth of blood vessels from pre-existing vasculature). The VEGF family includes VEGF-A, VEGF-B, VEGF-C, VEGF-D, P1GF (placental growth factor), VEGF-E (Orf-VEGF), and Trimeresurus flavoviridis svVEGF.

The term “VEGF receptor” or “VEGFR” as used herein refers to receptors for vascular endothelial growth factor (VEGF). There are three main subtypes of VEGFR, numbered 1, 2 and 3. The VEGF receptors may be membrane-bound or soluble, depending on alternative splicing. Among the VEGF receptors, VEGFR-1 binds VEGF-A, P1GF, and VEGF-B.

As used herein, a “bispecific antibody” refers to an artificial antibody, which has fragments derived from two different monoclonal antibodies and is capable of binding to two different epitopes. The two epitopes may present on the same antigen, or they may present on two different antigens.

The term “bispecific antigen-binding molecule” means a protein, polypeptide or molecular complex comprising at least a first antigen-binding domain (also referred to as a first antigen-binding site herein) and a second antigen-binding domain (also referred to as a second antigen-binding site herein). In some embodiments, the “bispecific antigen-binding molecule” is a “bispecific antibody”. Each antigen-binding domain within the bispecific antibody comprises at least one CDR that alone, or in combination with one or more additional CDRs and/or FRs, specifically binds to a particular antigen. In the context of the present disclosure, the first antigen-binding site specifically binds to a first antigen (e.g., PD-L1), and the second antigen-binding site specifically binds to a second, distinct antigen (e.g., VEGF).

The term “anti-PD-L1/anti-VEGF antibody”, “anti-PD-L1/anti-VEGF bispecific antibody”, “antibody against PD-L1 and VEGF”, “anti-PD-L1×VEGF bispecific antibody”, “PD-L1×VEGF antibody”, as used herein interchangeably, refers to a bispecific antibody that specifically binds to PD-L1 and VEGF.

The term “monoclonal antibody” or “mAb”, as used herein, refer to a preparation of antibody molecules of single molecular composition. A monoclonal antibody displays a single binding specificity and affinity for a particular epitope.

The term “human antibody”, as used herein, is intended to include antibodies having variable regions in which both the framework and CDR regions are derived from human germline immunoglobulin sequences. Furthermore, if the antibody contains a constant region, the constant region also is derived from human germline immunoglobulin sequences. The human antibodies can include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences derived from the germline of another mammalian species, such as a mouse, have been grafted onto human framework sequences.

The term “humanized antibody” is intended to refer to antibodies in which CDR sequences derived from the germline of another mammalian species, such as a mouse, have been grafted onto human framework sequences. Additional framework region modifications may be made within the human framework sequences.

The term “operably linked” refers to a juxtaposition, with or without a spacer or linker, of two or more biological sequences of interest in such a way that they are in a relationship permitting them to function in an intended manner. When used with respect to polypeptides, it is intended to mean that the polypeptide sequences are linked in such a way that permits the linked product to have the intended biological function. For example, an antibody variable region may be operably linked to a constant region so as to provide for a stable product with antigen-binding activity. The term may also be used with respect to polynucleotides. For one instance, when a polynucleotide encoding a polypeptide is operably linked to a regulatory sequence (e.g., promoter, enhancer, silencer sequence, etc.), it is intended to mean that the polynucleotide sequences are linked in such a way that permits regulated expression of the polypeptide from the polynucleotide. The term operably linked, when used herein to describe domains linked to form a polypeptide, can be represented by a “-”, and can refer to a direct linkage between domains or linkage via a linker comprising 1-30 amino acids in length, such as a single amino acid or a series of (G4S)n linker, with n=1-5 (1, 2, 3, 4, or 5).

The term “Ka,” as used herein, is intended to refer to the association rate of a particular antibody-antigen interaction, whereas the term “Kd” as used herein, is intended to refer to the dissociation rate of a particular antibody-antigen interaction. Kd values for antibodies can be determined using methods well established in the art. The term “K_{D}” as used herein, is intended to refer to the dissociation constant of a particular antibody-antigen interaction, which is obtained from the ratio of Kd to Ka (i.e., Kd/Ka) and is expressed as a molar concentration (M). A preferred method for determining the Kd of an antibody is by using surface plasmon resonance, preferably using a biosensor system such as a Biacore® system.

The term “high affinity” for an IgG antibody, as used herein, refers to an antibody having a K_{D} of 1×10⁻⁸ M or less, more preferably 5×10⁻⁹ M or less, even more preferably 1×10⁻⁹ M or less, even more preferably 5×10⁻¹⁰ M or less, even more preferably 4×10⁻¹⁰ M, even more preferably 3×10⁻¹⁰ M or less, even more preferably 2×10⁻¹⁰ M or less, even more preferably 1×10⁻¹⁰ M or less, even more preferably 5×10⁻¹¹ M or less, and even more preferably 3×10⁻¹¹ M or less for a target antigen.

The term “EC₅₀,” as used herein, which is also termed as “half maximal effective concentration” refers to the concentration of a drug, antibody or toxicant which induces a response halfway between the baseline and maximum after a specified exposure time. In the context of the application, EC₅₀ is expressed in the unit of “nM”.

The term “IC₅₀”, as used herein, which is also termed as “half maximal inhibitory concentration” is a measure of the potency of a substance in inhibiting a specific biological or biochemical function. In the context of the application, IC₅₀ is expressed in the unit of “nM”.

The ability of “inhibit binding,” as used herein, refers to the ability of an antibody or antigen-binding fragment thereof to inhibit the binding of two molecules (eg, human PD-L1 and PD-1, VEGFR1 and VEGF) to any detectable level. In certain embodiments, the binding of the two molecules can be inhibited by the antibodies at an IC₅₀ of no more than 50 nM, no more than 30 nM, no more than 10 nM, no more than 5 nM, no more than 1 nM or even less.

The term “isolated,” as used herein, refers to a state obtained from natural state by artificial means. If a certain “isolated” substance or component is present in nature, it is possible because its natural environment changes, or the substance is isolated from natural environment, or both. For example, a certain un-isolated polynucleotide or polypeptide naturally exists in a certain living animal body, and the same polynucleotide or polypeptide with a high purity isolated from such a natural state is called isolated polynucleotide or polypeptide. The term “isolated” excludes neither the mixed artificial or synthesized substance nor other impure substances that do not affect the activity of the isolated substance.

The term “isolated antibody,” as used herein, is intended to refer to an antibody that is substantially free of other antibodies having different antigenic specificities (e.g., an isolated antibody that specifically binds a PD-LT/VEGF protein is substantially free of antibodies that specifically bind antigens other than PD-LT/VEGF proteins). An isolated antibody that specifically binds a human PD-LT/VEGF protein may, however, have cross-reactivity to other antigens, such as PD-LT/VEGF proteins from other species. Moreover, an isolated antibody can be substantially free of other cellular material and/or chemicals.

The term “vector,” as used herein, refers to a nucleic acid vehicle which can have a polynucleotide inserted therein. When the vector allows for the expression of the protein encoded by the polynucleotide inserted therein, the vector is called an expression vector. The vector can have the carried genetic material elements expressed in a host cell by transformation, transduction, or transfection into the host cell. Vectors are well known by a person skilled in the art, including, but not limited to plasmids, phages, cosmids, artificial chromosome such as yeast artificial chromosome (YAC), bacterial artificial chromosome (BAC) or P1-derived artificial chromosome (PAC); phage such as λ phage or M13 phage and animal virus. The animal viruses that can be used as vectors, include, but are not limited to, retrovirus (including lentivirus), adenovirus, adeno-associated virus, herpes virus (such as herpes simplex virus), pox virus, baculovirus, papillomavirus, papova virus (such as SV40). A vector may comprise multiple elements for controlling expression, including, but not limited to, a promoter sequence, a transcription initiation sequence, an enhancer sequence, a selection element and a reporter gene. In addition, a vector may comprise origin of replication.

The term “host cell,” as used herein, refers to a cellular system which can be engineered to generate proteins, protein fragments, or peptides of interest. Host cells include, without limitation, cultured cells, e.g., mammalian cultured cells derived from rodents (rats, mice, guinea pigs, or hamsters) such as CHO, BHK, NSO, SP2/0, YB2/0; or human tissues or hybridoma cells, yeast cells, and insect cells, and cells comprised within a transgenic animal or cultured tissue. The term encompasses not only the particular subject cell but also the progeny of such a cell. Because certain modifications may occur in succeeding generations due to either mutation or environmental influences, such progeny may not be identical to the parent cell, but are still included within the scope of the term “host cell.”

The term “identity” or “homology”, as used herein, refers to a relationship between the sequences of two or more polypeptide molecules or two or more nucleic acid molecules, as determined by aligning and comparing the sequences. “Percent identity” means the percent of identical residues between the amino acids or nucleotides in the compared molecules and is calculated based on the size of the smallest of the molecules being compared. For these calculations, gaps in alignments (if any) are preferably addressed by a particular mathematical model or computer program (i.e., an “algorithm”). Methods that can be used to calculate the identity of the aligned nucleic acids or polypeptides include those described in Computational Molecular Biology, (Lesk, A. M., ed.), 1988, New York: Oxford University Press; Biocomputing Informatics and Genome Projects, (Smith, D. W., ed.), 1993, New York: Academic Press; Computer Analysis of Sequence Data, Part I, (Griffin, A. M., and Griffin, H. G., eds.), 1994, New Jersey: Humana Press; von Heinje, G., 1987, Sequence Analysis in Molecular Biology, New York: Academic Press; Sequence Analysis Primer, (Gribskov, M. and Devereux, J., eds.), 1991, New York: M. Stockton Press; and Carillo et al, 1988, SIAMJ. Applied Math. 48:1073.

The term “immunogenicity,” as used herein, refers to ability of stimulating the formation of specific antibodies or sensitized lymphocytes in organisms. It not only refers to the property of an antigen to stimulate a specific immunocyte to activate, proliferate and differentiate so as to finally generate immunologic effector substance such as antibody and sensitized lymphocyte, but also refers to the specific immune response that antibody or sensitized T lymphocyte can be formed in immune system of an organism after stimulating the organism with an antigen. Immunogenicity is the most important property of an antigen. Whether an antigen can successfully induce the generation of an immune response in a host depends on three factors, properties of an antigen, reactivity of a host, and immunization means.

The term “transfection,” as used herein, refers to the process by which nucleic acids are introduced into eukaryotic cells, particularly mammalian cells. Protocols and techniques for transfection include but not limited to lipid transfection and chemical and physical methods such as electroporation. A number of transfection techniques are well known in the art and are disclosed herein. See, e.g., Graham et al., 1973, Virology 52:456; Sambrook et al., 2001, Molecular Cloning: A Laboratory Manual, supra; Davis et al., 1986, Basic Methods in Molecular Biology, Elsevier; Chu et al, 1981, Gene 13:197. In a specific embodiment of the disclosure, human PD-L1/VEGF gene was transfected into 293F cells.

The term “SPR” or “surface plasmon resonance,” as used herein, refers to and includes an optical phenomenon that allows for the analysis of real-time biospecific interactions by detection of alterations in protein concentrations within a biosensor matrix, for example using the BIAcore system (Pharmacia Biosensor AB, Uppsala, Sweden and Piscataway, N.J.). For further descriptions, see Example 5 and Jansson, U., et al. (1993) Ann. Biol. Clin. 51:19-26; Jönsson, U., et al. (1991) Biotechniques 11:620-627; Johnsson, B., et al. (1995) J. Mol. Recognit. 8:125-131; and Johnnson, B., et al. (1991) Anal. Biochem. 198:268-277.

The term “fluorescence-activated cell sorting” or “FACS,” as used herein, refers to a specialized type of flow cytometry. It provides a method for sorting a heterogeneous mixture of biological cells into two or more containers, one cell at a time, based upon the specific light scattering and fluorescent characteristics of each cell (FlowMetric. “Sorting Out Fluorescence Activated Cell Sorting”. Retrieved 2017-11-09.). Instruments for carrying out FACS are known to those of skill in the art and are commercially available to the public. Examples of such instruments include FACS Star Plus, FACScan and FACSort instruments from Becton Dickinson (Foster City, Calif.) Epics C from Coulter Epics Division (Hialeah, Fla.) and MoFlo from Cytomation (Colorado Springs, Colo.).

The term “subject” includes any human or nonhuman animal, preferably humans.

The term “cancer,” as used herein, refers to any or a tumor or a malignant cell growth, proliferation or metastasis-mediated, solid tumors and non-solid tumors such as leukemia and initiate a medical condition.

The term “treatment,” “treating” or “treated,” as used herein in the context of treating a condition, pertains generally to treatment and therapy, whether of a human or an animal, in which some desired therapeutic effect is achieved, for example, the inhibition of the progress of the condition, and includes a reduction in the rate of progress, a halt in the rate of progress, regression of the condition, amelioration of the condition, and cure of the condition. For cancer, “treating” may refer to dampen or slow the tumor or malignant cell growth, proliferation, or metastasis, or some combination thereof. For tumors, “treatment” includes removal of all or part of the tumor, inhibiting or slowing tumor growth and metastasis, preventing or delaying the development of a tumor, or some combination thereof.

The term “prevent,” “prevention” or “preventing,” as used herein in the context of preventing a condition, refers generally to preventing or delaying the onset of the disease, or preventing the manifestation of clinical or subclinical symptoms thereof in a subject (whether a human or animal), for example, preventing the disease from occurring in a subject predisposed to the condition or disease but has not yet been diagnosed as having it.

The term “an effective amount,” as used herein, pertains to that amount of an active compound, or a material, composition or dosage from comprising an active compound, which is effective for producing some desired therapeutic effect, commensurate with a reasonable benefit/risk ratio, when administered in accordance with a desired treatment regimen. For instance, the “an effective amount,” when used in connection with treatment of PD-L1/VEGF-related diseases or conditions, refers to an antibody or antigen-binding portion thereof in an amount or concentration effective to treat the said diseases or conditions.

The term “pharmaceutically acceptable,” as used herein, means that the vehicle, diluent, excipient and/or salts thereof, are chemically and/or physically is compatible with other ingredients in the formulation, and the physiologically compatible with the recipient.

As used herein, the term “a pharmaceutically acceptable carrier and/or excipient” refers to a carrier and/or excipient pharmacologically and/or physiologically compatible with a subject and an active agent, which is well known in the art (see, e.g., Remington's Pharmaceutical Sciences. Edited by Gennaro A R, 19th ed. Pennsylvania: Mack Publishing Company, 1995), and includes, but is not limited to pH adjuster, surfactant, adjuvant and ionic strength enhancer. For example, the pH adjuster includes, but is not limited to, phosphate buffer; the surfactant includes, but is not limited to, cationic, anionic, or non-ionic surfactant, e.g., Tween-80; the ionic strength enhancer includes, but is not limited to, sodium chloride.

As used herein, the term “adjuvant” refers to a non-specific immunopotentiator, which can enhance immune response to an antigen or change the type of immune response in an organism when it is delivered together with the antigen to the organism or is delivered to the organism in advance. There are a variety of adjuvants, including, but not limited to, aluminium adjuvants (for example, aluminum hydroxide), Freund's adjuvants (for example, Freund's complete adjuvant and Freund's incomplete adjuvant), coryne bacterium parvum, lipopolysaccharide, cytokines, and the like. Freund's adjuvant is the most commonly used adjuvant in animal experiments now. Aluminum hydroxide adjuvant is more commonly used in clinical trials.

### Bispecific Antibodies and Antigen-Binding Portions Thereof

In one aspect, provided herein is a bispecific antibody and antigen-binding portions thereof. In some embodiments, the bispecific antibodies and antigen-binding portions thereof have a first specificity for PD-L1, and a second specificity for VEGF. In some further embodiments, the antibodies provided herein are multipecific.

According to certain exemplary embodiments, the present disclosure includes a bispecific antibody or the antigen-binding portion thereof, comprising a first antigen-binding moiety that specifically binds to PD-L1 and a second antigen-binding moiety that specifically binds to VEGF. Such antibodies may be referred to herein as, e.g., “anti-VEGF/anti-PD-L1” or “anti-PD-L1/VEGF” or “anti-PD-L1×VEGF” or “PD-L1×VEGF” bispecific antibodies, or other similar terminology.

In some embodiments, the bispecific antibody comprises a PD-L1 binding moiety derived from a parental anti-PD-L1 antibody operably linked to an anti-VEGF antibody. In some other embodiments, the bispecific antibody comprises a VEGF binding moiety derived from a parental anti-VEGF antibody operably linked to an anti-PD-L1 antibody. In some other embodiments, the bispecific antibody comprises a PD-L1 binding moiety derived from a parental anti-PD-L1 antibody operably linked to a VEGF binding moiety derived from a parental anti-VEGF antibody.

The bispecific antibodies of the disclosure could bind to human PD-L1 and human VEGF with high affinity. The binding of an antibody of the disclosure to PD-L1 or VEGF can be assessed using one or more techniques well established in the art, for instance, ELISA. In an ELISA assy, a recombinant PD-L1 protein may be used. The binding specificity of the antibody disclosed herein can also be determined by monitoring binding of the antibody to cells expressing a PD-L1 protein or a VEGF protein, e.g., flow cytometry. For example, an antibody can be tested by a flow cytometry assay in which the antibody is reacted with a cell line that expresses human PD-L1, such as CHO cells that have been transfected to express PD-L1 on their cell surface. Additionally or alternatively, the binding of the antibody, including the binding kinetics (e.g., K_{D} value) can be tested in BIAcore binding assays.

For instance, an antibody of the disclosure binds to a human PD-L1 protein or human VEGF protein with a K_{D} of 1×10⁻⁸ M or less, a K_{D} of 5×10⁻⁹ M or less, a K_{D} of 2×10⁻⁹ M or less, a K_{D} of 1×10⁻⁹ M or less, a K_{D} of 5×10⁻¹⁰ M or less, a K_{D} of 4×10⁻¹⁰ M or less, a K_{D} of 3×10⁻¹⁰ M or less, a K_{D} of 2×10⁻¹⁰ M or less, a K_{D} of 1×10⁻¹⁰ M or less, a K_{D} of 5×10⁻¹¹ M or less, or a K_{D} of 3×10⁻¹¹ M or less, as measured by Surface Plasmon Resonance.

As demonstrated in the Example section, the bispecific antibodies of the disclosure could bind to PD-L1 and VEGF with a high affinity; effectively block both PD-1/PD-L1 and VEGFR/VEGF signaling pathways, e.g. with an IC50 of nM grade; block VEGF induced HUVEC proliferation; and produce strong agonistic effect on cytokine secretion.

In some embodiments, the bispecific antibodies as disclosed herein have a higher binding affinity to PD-L1 as compared to monospecific anti-PD-L1 antibodies (e.g. atezolizumab) or other anti-PD-L1/VEGF bispecific antibodies. In some embodiments, the bispecific antibodies as disclosed herein have binding affinity to PD-L1 that is at least 10%, 20%, 30%, 40%, 50%, 60%, 70%, 80%, 90%, or 100% higher than monospecific anti-PD-L1 antibodies or other anti-PD-L1/VEGF bispecific antibodies, e.g. as measured in K_{D}.

In some embodiments, the bispecific antibodies as disclosed herein have a higher binding affinity to VEGF as compared to monospecific anti-VEGF antibodies (e.g. Avastin) or other anti-PD-L1/VEGF bispecific antibodies. In some embodiments, the bispecific polypeptide complexes as disclosed herein have binding affinity to VEGF that is at least 10%, 20%, 30%, 40%, 50%, 60%, 70%, 80%, 90%, or 100% higher than monospecific anti-VEGF antibodies or other anti-PD-L1/VEGF bispecific antibodies, e.g. as measured in K_{D}.

In some embodiments, the bispecific antibodies as disclosed herein have higher binding affinity to PD-L1 as compared to monospecific anti-PD-L1 antibodies (e.g. atezolizumab) and/or higher binding affinity to VEGF as compared to monospecific anti-VEGF antibodies (e.g. Avastin). In some embodiments, the bispecific antibodies as disclosed herein have a higher binding affinity to PD-L1 as compared to atezolizumab, and higher or comparable binding affinity to VEGF as compared to Avastin. In some embodiments, the bispecific antibodies as disclosed herein have significantly improved anti-tumor efficacy compared to a combination of monospecific anti-PD-L1 antibodies and monospecific anti-VEGF antibodies, such as a combination of atezolizumab and Avastin.

In some embodiments, the bispecific antibodies as disclosed herein have higher binding affinity to PD-L1 as compared to other anti-PD-L1/VEGF bispecific antibodies and/or higher binding affinity to VEGF as compared to other anti-PD-L1/VEGF bispecific antibodies.

### The PD-L1 Antigen-Binding Moiety

The PD-L1 binding moiety as defined herein may be presented in various formats (e.g. VHH, scFv, Fab), as long as it can fit into the bispecific antibody and specifically bind to PD-L1. Generally, the PD-L1 binding moiety comprised in the bispecific antibody is derived from a monospecific anti-PD-L1 antibody, which may be a known antibody or an antibody developed de novo. In some embodiments according to the present application, the PD-L1 binding moiety is derived from a fully human monoclonal antibody. Various methods for obtaining fully human antibodies against a specific antigen are known to these skilled in the art, for example by immunizing a transgenic non-human animal (e.g. OMT rat) that utilizes human antibody repertoires or other human antibody-encoding sequences.

The PD-L1 binding moiety may be derived from an anti-PD-L1 monoclonal antibody. By “derive from” it is meant herein that the PD-L1 binding moiety comprises or consists of the antigen-binding portion of the PD-L1 or a variant thereof which retains the antigen binding ability. For example, the PD-L1 binding moiety may be a scFv, Fab, or VHH fragment of the anti-PD-L1 parental antibody.

In some embodiments, the PD-L1 binding moiety comprises a single chain Fv fragment (scFv) comprising a VH of an anti-PD-L1 antibody operably linked to a VL of the anti-PD-L1 antibody. The VH and VL may be linked via a peptide linker such as (G4S)n, with n=1 to 4.

In some embodiments, the PD-L1 antigen binding moiety comprises one or more CDRs selected from the group consisting of:
(i) a HCDR1 comprising the amino acid sequence of SEQ ID NO: 1 or an amino acid sequence that differs from SEQ ID NO: 1 by an amino acid addition, deletion or substitution of not more than 1, 2 or 3 amino acids;
(ii) a HCDR2 comprising the amino acid sequence of SEQ ID NO: 2 or an amino acid sequence that differs from SEQ ID NO: 2 by an amino acid addition, deletion or substitution of not more than 1, 2 or 3 amino acids;
(iii) a HCDR3 comprising the amino acid sequence of SEQ ID NO: 3 or an amino acid sequence that differs from SEQ ID NO: 3 by an amino acid addition, deletion or substitution of not more than 1, 2 or 3 amino acids;
(iv) a LCDR1 comprising the amino acid sequence of SEQ ID NO: 4 or an amino acid sequence that differs from SEQ ID NO: 4 by an amino acid addition, deletion or substitution of not more than 1, 2 or 3 amino acids;
(v) a LCDR2 comprising the amino acid sequence of SEQ ID NO: 5 or an amino acid sequence that differs from SEQ ID NO: 5 by an amino acid addition, deletion or substitution of not more than 1, 2 or 3 amino acids; and
(vi) a LCDR3 comprising the amino acid sequence of SEQ ID NO: 6 or an amino acid sequence that differs from SEQ ID NO: 6 by an amino acid addition, deletion or substitution of not more than 1, 2 or 3 amino acids.

In some embodiments, the VH comprises (i) a HCDR1 comprising or consisting of the amino acid sequence of SEQ ID NO: 1; (ii) a HCDR2 comprising or consisting of the amino acid sequence of SEQ ID NO: 2; and (iii) a HCDR3 comprising or consisting of the amino acid sequence of SEQ ID NO: 3; and the VL comprises: (i) a LCDR1 comprising or consisting of the amino acid sequence of SEQ ID NO: 4; (ii) a LCDR2 comprising or consisting of the amino acid sequence of SEQ ID NO: 5; and (iii) a LCDR3 comprising or consisting of the amino acid sequence of SEQ ID NO: 6.

In some embodiments, the PD-L1 antigen-binding moiety comprises a VH and a VL region, wherein the VH region comprises: (i) the amino acid sequence of SEQ ID NO: 13; (ii) an amino acid sequence with at least 85%, 90%, or 95% identity (preferably, at least 90%, more preferably, at least 95% (e.g., 95%, 96%, 97%, 98%, or 99%) identity) to SEQ ID NO: 13; or (iii) an amino acid sequence with addition, deletion and/or substitution of one or more (e.g. 10, 9, 8, 7, 6, 5, 4, 3, 2, 1) amino acid(s) compared with SEQ ID NO: 13; and/or
the VL region comprises: (i) the amino acid sequence of SEQ ID NO: 14; (ii) an amino acid sequence with at least 85%, 90%, or 95% identity (preferably, at least 90%, more preferably, at least 95% (e.g., 95%, 96%, 97%, 98%, or 99%) identity) to SEQ ID NO: 14; or (iii) an amino acid sequence with addition, deletion and/or substitution of one or more (e.g. 10, 9, 8, 7, 6, 5, 4, 3, 2, 1) amino acid(s) compared with SEQ ID NO: 14.

### The VEGF Antigen-Binding Moiety

Similarly, the VEGF antigen-binding moiety provided herein may be derived from a parental anti-VEGF monospecific antibody. In some embodiments according to the present application, the VEGF antigen-binding moiety is the Fab fragment of an anti-VEGF full antibody, i.e. comprising VH region and CH1 region in the heavy chain, and VL region and CL region in the light chain.

The anti-VEGF antibody which is used as the parental antibody may be a monoclonal antibody already known in the art (such as Bevacizumab) or which is de novo developed. Preferably, the anti-VEGF antibody is a fully human antibody or a humanized antibody.

In some embodiments, the VEGF binding moiety is derived from a fully human monoclonal antibody. For example, the VEGF binding moiety may be a scFv, Fab, or VHH fragment of the anti-VEGF parental antibody. In some embodiments, the VEGF binding moiety comprises a Fab comprising a VH of an anti-VEGF antibody associated with a VL of the anti-VEGF antibody.

In some embodiments, the VH region of the VEGF antigen-binding moiety comprises one or more heavy chain CDRs (HCDRs) selected from the group consisting of:
(i) a HCDR1 comprising the amino acid sequence of SEQ ID NO: 7 or an amino acid sequence that differs from SEQ ID NO: 7 by an amino acid addition, deletion or substitution of not more than 1, 2 or 3 amino acids;
(ii) a HCDR2 comprising the amino acid sequence of SEQ ID NO: 8 or an amino acid sequence that differs from SEQ ID NO: 8 by an amino acid addition, deletion or substitution of not more than 1, 2 or 3 amino acids; and
(iii) a HCDR3 comprising the amino acid sequence of SEQ ID NO: 9 or an amino acid sequence that differsfrom SEQ ID NO: 9 by an amino acid addition, deletion or substitution of not more than 1, 2 or 3 amino acids; and/or the VL region comprises one or more light chain CDRs (LCDRs) selected from the group consisting of:
(i) a LCDR1 comprising the amino acid sequence of SEQ ID NO: 10 or an amino acid sequence that differs from SEQ ID NO: 10 by an amino acid addition, deletion or substitution of not more than 1, 2 or 3 amino acids;
(ii) a LCDR2 comprising the amino acid sequence of SEQ ID NO: 11 or an amino acid sequence that differs from SEQ ID NO: 11 by an amino acid addition, deletion or substitution of not more than 1, 2 or 3 amino acids; and
(iii) a LCDR3 comprising the amino acid sequence of SEQ ID NO: 12 or an amino acid sequence that differs from SEQ ID NO: 12 by an amino acid addition, deletion or substitution of not more than 1, 2 or 3 amino acids.

In some embodiments, the VH comprises (i) a HCDR1 comprising or consisting of the amino acid sequence of SEQ ID NO: 7; (ii) a HCDR2 comprising or consisting of the amino acid sequence of SEQ ID NO: 8; and (iii) a HCDR3 comprising or consisting of the amino acid sequence of SEQ ID NO: 9; and the VL comprises: (i) a LCDR1 comprising or consisting of the amino acid sequence of SEQ ID NO: 10; (ii) a LCDR2 comprising or consisting of the amino acid sequence of SEQ ID NO: 11; and (iii) a LCDR3 comprising or consisting of the amino acid sequence of SEQ ID NO: 12.

In some embodiments, the VH of the VEGF antigen-binding moiety comprises: (i) the amino acid sequence of SEQ ID NO: 15; (ii) an amino acid sequence with at least 85%, 90%, or 95% identity (preferably, at least 90%, more preferably, at least 95% (e.g., 95%, 96%, 97%, 98%, or 99%) identity) to SEQ ID NO: 15; or (iii) an amino acid sequence with addition, deletion and/or substitution of one or more (e.g. 10, 9, 8, 7, 6, 5, 4, 3, 2, 1) amino acids compared with SEQ ID NO: 15.

In some embodiments, the VL of the VEGF antigen-binding moiety comprises: (i) the amino acid sequence of SEQ ID NO: 16; (ii) an amino acid sequence with at least 85%, 90%, or 95% identity (preferably, at least 90%, more preferably, at least 95% (e.g., 95%, 96%, 97%, 98%, or 99%) identity) to SEQ ID NO: 16; or (iii) an amino acid sequence with addition, deletion and/or substitution of one or more (e.g. 10, 9, 8, 7, 6, 5, 4, 3, 2, 1) amino acids compared with SEQ ID NO: 16.

The assignment of amino acids to each CDR may be in accordance with one of the numbering schemes provided by Kabat et al. (1991) Sequences of Proteins of Immunological Interest (5^{th} Ed.), US Dept. of Health and Human Services, PHS, NIH, NIH Publication no. 91-3242; Chothia et al., 1987, PMID: 3681981; Chothia et al., 1989, PMID: 2687698; MacCallum et al., 1996, PMID: 8876650; or Dubel, Ed. (2007) Handbook of Therapeutic Antibodies, 3^{rd} Ed., Wily-VCH Verlag GmbH and Co. unless otherwise noted.

Variable regions and CDRs in an antibody sequence can be identified according to general rules that have been developed in the art (as set out above, such as, for example, the Kabat numbering system) or by aligning the sequences against a database of known variable regions. Methods for identifying these regions are described in Kontermann and Dubel, eds., Antibody Engineering, Springer, New York, NY, 2001 and Dinarello et al., Current Protocols in Immunology, John Wiley and Sons Inc., Hoboken, NJ, 2000. Exemplary databases of antibody sequences are described in, and can be accessed through, the “Abysis” website at www.bioinf.org.uk/abs (maintained by A. C. Martin in the Department of Biochemistry & Molecular Biology University College London, London, England) and the VBASE2 website at www.vbase2.org, as described in Retter et al., Nucl. Acids Res., 33 (Database issue): D671-D674 (2005). Preferably sequences are analyzed using the Abysis database, which integrates sequence data from Kabat, IMGT and the Protein Data Bank (PDB) with structural data from the PDB. See Dr. Andrew C. R. Martin's book chapter Protein Sequence and Structure Analysis of Antibody Variable Domains. In: Antibody Engineering Lab Manual (Ed.: Duebel, S. and Kontermann, R., Springer-Verlag, Heidelberg, ISBN-13: 978-3540413547, also available on the website bioinforg.uk/abs). The Abysis database website further includes general rules that have been developed for identifying CDRs which can be used in accordance with the teachings herein. Unless otherwise indicated, all CDRs set forth herein are derived according to the Abysis database website as per Kabat.

The percent identity between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percentage of identity between two amino acid sequences can be determined by the algorithm of Needleman and Wunsch (J. Mol. Biol. 48:444-453 (1970)) which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6.

Additionally or alternatively, the protein sequences of the present disclosure can further be used as a “query sequence” to perform a search against public databases to, for example, identify related sequences. Such searches can be performed using the XBLAST program (version 2.0) of Altschul, et al. (1990) J. Mol. Biol. 215:403-10. BLAST protein searches can be performed with the XBLAST program, score=50, wordlength=3 to obtain amino acid sequences homologous to the antibody molecules of the disclosure. To obtain gapped alignments for comparison purposes, Gapped BLAST can be utilized as described in Altschul et al, (1997) Nucleic Acids Res. 25(17):3389-3402. When utilizing BLAST and Gapped BLAST programs, the default parameters of the respective programs (e.g., XBLAST and NBLAST) can be used. See www.ncbi.nlm.nih.gov.

In other embodiments, the CDR amino acid sequences can be at least 90%, 91%, 92%, 93%, 94%, 95%, 96%, 97%, 98% or 99% identical to the respective sequences set forth above. In other embodiments, the amino acid sequences of the variable region can be at least 90%, 91%, 92%, 93%, 94%, 95%, 96%, 97%, 98% or 99% identical to the respective sequences set forth above.

Preferably, the CDRs of the isolated antibody or the antigen-binding portion thereof contain a conservative substitution of not more than 2 amino acids, or not more than 1 amino acid. The term “conservative substitution”, as used herein, refers to amino acid substitutions which would not disadvantageously affect or change the essential properties of a protein/polypeptide comprising the amino acid sequence. For example, a conservative substitution may be introduced by standard techniques known in the art such as site-directed mutagenesis and PCR-mediated mutagenesis. Conservative amino acid substitutions include substitutions wherein an amino acid residue is substituted with another amino acid residue having a similar side chain, for example, a residue physically or functionally similar (such as, having similar size, shape, charge, chemical property including the capability of forming covalent bond or hydrogen bond, etc.) to the corresponding amino acid residue. The families of amino acid residues having similar side chains have been defined in the art. These families include amino acids having alkaline side chains (for example, lysine, arginine and histidine), amino acids having acidic side chains (for example, aspartic acid and glutamic acid), amino acids having uncharged polar side chains (for example, glycine, asparagine, glutamine, serine, threonine, tyrosine, cysteine, tryptophan), amino acids having nonpolar side chains (for example, alanine, valine, leucine, isoleucine, proline, phenylalanine, methionine), amino acids having β-branched side chains (such as threonine, valine, isoleucine) and amino acids having aromatic side chains (for example, tyrosine, phenylalanine, tryptophan, histidine). Therefore, a corresponding amino acid residue is preferably substituted with another amino acid residue from the same side-chain family. Methods for identifying amino acid conservative substitutions are well known in the art (see, for example, Brummell et al., Biochem. 32: 1180-1187 (1993); Kobayashi et al., Protein Eng. 12(10): 879-884 (1999); and Burks et al., Proc. Natl. Acad. Sci. USA 94: 412-417 (1997), which are incorporated herein by reference).

### Generation of Bispecific Antibodies

For constructing an anti-PD-LT/VEGF bispecific antibody, the PD-L1 antigen-binding moiety and the VEGF antigen-binding moiety as described above may be fused together in various formats. The PD-L1 antigen-binding moiety and VEGF antigen-binding moiety of the bispecific antibody may be directly or indirectly connected to one another. In certain embodiments, the PD-L1 antigen-binding moiety and the VEGF antigen-binding moiety may be connected to one another by a linker. The linker may be a peptide linker, such as comprising 1-4 copies of GGGGS (G4S). In one embodiment, the linker is (G4S)₂.

In some specific embodiments, the PD-L1 antigen-binding moiety is fused to the N terminal of the VEGF antigen-binding moiety. When the PD-L1 antigen-binding moiety is a scFv, the single chain of the PD-L1 antigen-binding moiety may be operably linked to the heavy chain or light chain of the VEGF antigen-binding moiety, optionally via a linker. Preferably, the PD-L1 antigen-binding moiety is linked to the heavy chain of the VEGF antigen-binding moiety via a peptide linker.

In certain embodiments, the fusion of the PD-L1 antigen-binding moiety and VEGF antigen-binding moiety is further bound to an Fc region. Alternatively, the PD-L1 moiety and the VEGF moiety is each connected to one end of an Fc region.

The bispecific antibodies and antigen-binding portions provided herein can be made with any suitable methods known in the art. In a conventional approach, two immunoglobulin heavy chain-light chain pairs can be co-expressed in a host cell to produce bispecific antibodies in a recombinant way (see, for example, Milstein and Cuello, Nature, 305: 537 (1983)), followed by purification by affinity chromatography. A different recombinant approach may also be used, where sequences encoding the antibody heavy chain variable domains for the two specificities are respectively fused to immunoglobulin constant domain sequences, followed by insertion to an expression vector which is co-transfected with an expression vector for the light chain sequences to a suitable host cell for recombinant expression of the bispecific antibody (see, for example, WO 94/04690; Suresh et al., Methods in Enzymology, 121:210 (1986)). Similarly, scFv dimers can also be recombinantly constructed and expressed from a host cell (see, e.g. Gruber et al., J. Immunol., 152:5368 (1994).)

### Fc Region

In certain embodiments, the Fc region is operably linked to the VEGF antigen-binding moiety. The Fc region of the bispecific antibodies disclosed herein is preferably a human IgG Fc region. The IgG Fc region may be of any isotype, including, but not limited to, IgG1, IgG2, IgG3 or IgG4. In certain embodiments, the Fc region is of the IgG1 isotype. Specifically, the heavy chain of the bispecific antibody may comprise domains operably linked as in scFv-VH-CH1-hinge-Fc, wherein the scFv is from the PD-L1 antigen-binding moiety and the VH-CH1 is from the VEGF antigen-binding moiety; and the light chain comprises domains operably linked as in VL-CL, wherein the VL-CL is from the VEGF antigen-binding moiety.

In the context of bispecific antibodies of the present disclosure, the Fc region may comprise one or more amino acid changes (e.g., insertions, deletions or substitutions) as compared to the specified chimeric version of the Fc region. The disclosure encompasses bispecific antigen-binding molecules comprising one or more modifications in the Fc region that results in a modified Fc region having a modified binding interaction between Fc and FcRn or FcγR.

For example, the Fc region may comprise one or more amino acid modification (e.g. Leu234Ala/Leu235Ala or LALA) that alters the antibody-dependent cellular cytotoxicity (ADCC) or other effector functions.

In certain embodiments, the Fc modification comprise a LALA mutation, i.e. mutations of L234A and L235A, according to EU numbering as in Kabat et al., LALA mutation is perhaps the most commonly used mutation for disrupting antibody effector function, e.g. eliminate Fc binding to specific FcγRs, reduce ADCC activity mediated by PBMCs and monocytes. The “EU numbering system” or “EU index” is generally used when referring to a residue in an immunoglobulin heavy chain constant region (e.g., the EU index reported in Kabat et al., supra). The “EU numbering as in Kabat” or “EU index as in Kabat” refers to the residue numbering of the human IgG1 EU antibody. Unless stated otherwise herein, references to residue numbers in the constant domain of antibodies means residue numbering by the EU numbering system.

In certain embodiments, the Fc region is operably linked to the VEGF binding moiety via a hinge region, which may be derived from human IgG1, IgG2 or IgG4. In certain embodiments, the hinge region has the same isotype as Fc and is also derived from human IgG1.

### Nucleic Acid Molecules Encoding Antibodies of the Disclosure

In some aspects, the disclosure is directed to an isolated nucleic acid molecule, comprising a nucleic acid sequence encoding the bispecific antibody or the antigen-binding portion as disclosed herein. For example, the nucleic acid sequence may encode a heavy chain and/or a light chain of the bispecific antibody. Alternatively, the nucleic acid sequence may encode the PD-L1 antigen-binding moiety, or a heavy chain or a light chain variable region of the VEGF antigen-binding moiety. The nucleic acid sequence may further encode the Fc region of the bispecific antibody.

In some embodiments, the isolated nucleic acid molecule comprises one or more nucleic acid sequence(s) selected from the group consisting of:
(A) a nucleic acid sequence that encodes the heavy chain sequence or light chain sequence of the VEGF-binding moiety;
(B) a nucleic acid sequence that encodes the amino acid sequene of the PD-L1-binding moiety;
(C) a nucleic acid sequence that encodes the heavy chain sequence of the VEGF-binding moiety operably linked to the amino acid sequence of the PD-L1-binding moiety;
(D) any combinations of (A)-(C); and
(E) a nucleic acid sequence that hybridized under high stringency conditions to the complementary strand of the nucleic acid sequence of (A)-(D).

In some embodiments, the nucleic acid sequence encoding the heavy chain of the bispecific antibody is as shown in SEQ ID NO: 19 and the nucleic acid sequence encoding the light chain of the bispecific antibody is as shown in SEQ ID NO: 20.

In some aspects, the disclosure is directed to a vector comprising the nucleic acid sequence as disclosed herein. In a further embodiment, the expression vector further comprises a nucleotide sequence encoding the constant region of a bispecific antibody, e.g. a humanized bispecific antibody.

A vector in the context of the present disclosure may be any suitable vector, including chromosomal, non-chromosomal, and synthetic nucleic acid vectors (a nucleic acid sequence comprising a suitable set of expression control elements). Examples of such vectors include derivatives of SV40, bacterial plasmids, phage DNA, baculovirus, yeast plasmids, vectors derived from combinations of plasmids and phage DNA, and viral nucleic acid (RNA or DNA) vectors. In one embodiment, a PD-L1 or a VEGF antibody-encoding nucleic acid is comprised in a naked DNA or RNA vector, including, for example, a linear expression element (as described in for instance Sykes and Johnston, Nat Biotech 17, 355-59 (1997)), a compacted nucleic acid vector (as described in for instance U.S. Pat. No. 6,077,835 and/or WO 00/70087), a plasmid vector such as pBR322, pUC 19/18, or pUC 118/119, a “midge” minimally-sized nucleic acid vector (as described in for instance Schakowski et al., Mol Ther 3, 793-800 (2001)), or as a precipitated nucleic acid vector construct, such as a CaP04-precipitated construct (as described in for instance WO200046147, Benvenisty and Reshef, PNAS USA 83, 9551-55 (1986), Wigler et al., Cell 14, 725 (1978), and Coraro and Pearson, Somatic Cell Genetics 7, 603 (1981)). Such nucleic acid vectors and the usage thereof are well known in the art (see for instance U.S. Pat. Nos. 5,589,466 and 5,973,972).

In one embodiment, the vector is suitable for expression of the anti-PD-L1 antibody and/or anti-VEGF antibody in a bacterial cell. Examples of such vectors include expression vectors such as BlueScript (Stratagene), pIN vectors (Van Heeke & Schuster, J Biol Chem 264, 5503-5509 (1989), pET vectors (Novagen, Madison WI) and the like). A vector may also or alternatively be a vector suitable for expression in a yeast system. Any vector suitable for expression in a yeast system may be employed. Suitable vectors include, for example, vectors comprising constitutive or inducible promoters such as alpha factor, alcohol oxidase and PGH (reviewed in: F. Ausubel et al., ed. Current Protocols in Molecular Biology, Greene Publishing and Wiley InterScience New York (1987), and Grant et al., Methods in Enzymol 153, 516-544 (1987)).

A vector may also or alternatively be a vector suitable for expression in mammalian cells, e.g. a vector comprising glutamine synthetase as a selectable marker, such as the vectors described in Bebbington (1992) Biotechnology (NY) 10: 169-175.

A nucleic acid and/or vector may also comprise a nucleic acid sequence encoding a secretion/localization sequence, which can target a polypeptide, such as a nascent polypeptide chain, to the periplasmic space or into cell culture media. Such sequences are known in the art, and include secretion leader or signal peptides.

The vector may comprise or be associated with any suitable promoter, enhancer, and other expression-facilitating elements. Examples of such elements include strong expression promoters (e. g., human CMV IE promoter/enhancer as well as RSV, SV40, SL3-3, MMTV, and HIV LTR promoters), effective poly (A) termination sequences, an origin of replication for plasmid product in E. coli, an antibiotic resistance gene as selectable marker, and/or a convenient cloning site (e.g., a polylinker). Nucleic acids may also comprise an inducible promoter as opposed to a constitutive promoter such as CMV IE.

In a further aspect, the disclosure relates to a host cell comprising the vector specified herein above. Thus, the present disclosure also relates to a recombinant eukaryotic or prokaryotic host cell which produces a bispecific antibody of the present disclosure, such as a transfectoma.

The PD-L1-specific antibody may be expressed in a recombinant eukaryotic or prokaryotic host cell, such as a transfectoma, which produces an antibody of the disclosure as defined herein or a bispecific antibody of the disclosure as defined herein. The VEGF-specific antibody may likewise be expressed in a recombinant eukaryotic or prokaryotic host cell, such as a transfectoma, which produces an antibody of the disclosure as defined herein or a bispecific antibody of the disclosure as defined herein.

Examples of host cells include yeast, bacterial, plant and mammalian cells, such as CHO, CHO-S, HEK, HEK293, HEK-293F, Expi293F, PER.C6 or NSO cells or lymphocytic cells. For example, in one embodiment, the host cell may comprise a first and second nucleic acid construct stably integrated into the cellular genome. In another embodiment, the present disclosure provides a cell comprising a non-integrated nucleic acid, such as a plasmid, cosmid, phagemid, or linear expression element, which comprises a first and second nucleic acid construct as specified above.

Mammalian host cells for expressing the antibodies of the present disclosure include, but not limited to, Chinese Hamster Ovary (CHO cells) (including dhfr CHO cells, described in Urlaub and Chasm, (1980) Proc. Natl. Acad. ScL USA 77:4216-4220, used with a DHFR selectable marker, e.g., as described in R. J. Kaufman and P. A. Sharp (1982) J. MoI. Biol. 159:601-621), COS cells and SP2 cells. In particular, for use with NSO myeloma cells, another expression system is the GS gene expression system disclosed in WO 87/04462, WO 89/01036 and EP 338,841. Also included are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/-DHFR (CHO, Urlaub et al., 1980, Proc. Natl. Acad. Sci. USA 77:4216); mouse sertoli cells (TM4, Mather, 1980, Biol. Reprod. 23:243-251); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., 1982, Annals N.Y. Acad. Sci. 383:44-68); MRC 5 cells; FS4 cells; mouse myeloma cells, such as NSO (e.g. RCB0213, 1992, Bio/Technology 10:169) and SP2/0 cells (e.g. SP2/0-Ag14 cells, ATCC CRL 1581); rat myeloma cells, such as YB2/0 cells (e.g. YB2/3HL.P2.G11.16Ag.20 cells, ATCC CRL 1662); PER.C6 cells; and a human hepatoma line (Hep G2). CHO cells are one of the cell lines that can be used herein, with CHO-K1, DUK-B11, CHO-DP12, CHO-DG44 (Somatic Cell and Molecular Genetics 12:555 (1986)), and Lec13 being exemplary host cell lines. In the case of CHO-K1, DUK-B11, DG44 or CHO-DP12 host cells, these may be altered such that they are deficient in their ability to fucosylate proteins expressed therein.

Suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis, Pseudomonas such as P. aeruginosa, and Streptomyces.

In addition to prokaryotes, eukaryotic microbes such as filamentous fungi or yeast are suitable cloning or expression hosts for bispecific antibody-encoding vectors. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g., K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus yarrowia (EP 402,226); Pichia pastoris (EP 183,070); Candida; Trichoderma reesia (EP 244,234); Neurosporacrassa; Schwanniomyces such as Schwanniomycesoccidentalis; and filamentous fungi such as, e.g., Neurospora, Penicillium, Tolypocladium, and Aspergillus hosts such as A. nidulans and A. niger.

In a further aspect, the disclosure relates to a transgenic non-human animal or plant comprising nucleic acids encoding one or two sets of a human heavy chain and a human light chain, wherein the animal or plant produces a bispecific antibody of the disclosure.

In a further aspect, the disclosure relates to a hybridoma which produces an antibody for use in a bispecific antibody of the disclosure as defined herein.

In one aspect, the disclosure relates to an expression vector comprising:
(i) a nucleic acid sequence encoding the PD-L1 antigen-binding moiety;
(ii) a nucleic acid sequence encoding the heavy chain and/or the light chain of the VEGF antigen-binding moiety;
(iii) a nucleic acid sequence encoding a Fc region; or
(iv) a nucleic acid sequence encoding the heavy chain or the light chain of the bispecific antibody.

In one aspect, the disclosure relates to a method for producing a bispecific antibody according to any one of the embodiments as disclosed herein, comprising the steps of culturing a host cell as disclosed herein comprising an expression vector or more than one expression vectors expressing the bispecific antibody as disclosed herein and purifying said antibody from the culture media. In one aspect, the disclosure relates to a host cell comprising an expression vector as defined above. In one embodiment, the host cell is a recombinant eukaryotic, recombinant prokaryotic, or recombinant microbial host cell.

### Pharmaceutical Compositions

In some aspects, the disclosure is directed to a pharmaceutical composition comprising a bispecific antibody or antigen-binding portion thereof as disclosed herein and a pharmaceutically acceptable carrier.

### Components of the Compositions

The pharmaceutical composition may optionally contain one or more additional pharmaceutically active ingredients, such as another antibody or a drug. The pharmaceutical compositions of the disclosure also can be administered in a combination therapy with, for example, another immune-stimulatory agent, anti-cancer agent, an antiviral agent, or a vaccine, such that the anti-PD-LT/anti-VEGF bispecific antibody enhances the immune response against the vaccine. A pharmaceutically acceptable carrier can include, for example, a pharmaceutically acceptable liquid, gel or solid carriers, an aqueous medium, a non-aqueous medium, an antimicrobial agent, isotonic agents, buffers, antioxidants, anesthetics, suspending/dispersing agent, a chelating agent, a diluent, adjuvant, excipient or a nontoxic auxiliary substance, other known in the art various combinations of components or more.

Suitable components may include, for example, antioxidants, fillers, binders, disintegrating agents, buffers, preservatives, lubricants, flavorings, thickening agents, coloring agents, emulsifiers or stabilizers such as sugars and cyclodextrin. Suitable anti-oxidants may include, for example, methionine, ascorbic acid, EDTA, sodium thiosulfate, platinum, catalase, citric acid, cysteine, mercapto glycerol, thioglycolic acid, Mercapto sorbitol, butyl methyl anisole, butylated hydroxy toluene and/or propylgalacte. As disclosed in the present disclosure, in a solvent containing an antibody or an antigen-binding fragment of the present disclosure discloses compositions include one or more anti-oxidants such as methionine, reducing antibody or antigen binding fragment thereof may be oxidized. The oxidation reduction may prevent or reduce a decrease in binding affinity, thereby enhancing antibody stability and extended shelf life. Thus, in some embodiments, the present disclosure provides a composition comprising one or more antibodies or antigen binding fragment thereof and one or more anti-oxidants such as methionine. The present disclosure further provides a variety of methods, wherein an antibody or antigen binding fragment thereof is mixed with one or more anti-oxidants, such as methionine, so that the antibody or antigen binding fragment thereof can be prevented from oxidation, to extend their shelf life and/or increased activity.

To further illustrate, pharmaceutical acceptable carriers may include, for example, aqueous vehicles such as sodium chloride injection, Ringer's injection, isotonic dextrose injection, sterile water injection, or dextrose and lactated Ringer's injection, nonaqueous vehicles such as fixed oils of vegetable origin, cottonseed oil, corn oil, sesame oil, or peanut oil, antimicrobial agents at bacteriostatic or fungistatic concentrations, isotonic agents such as sodium chloride or dextrose, buffers such as phosphate or citrate buffers, antioxidants such as sodium bisulfate, local anesthetics such as procaine hydrochloride, suspending and dispersing agents such as sodium carboxymethylcelluose, hydroxypropyl methylcellulose, or polyvinylpyrrolidone, emulsifying agents such as Polysorbate 80 (TWEEN-80), sequestering or chelating agents such as EDTA (ethylenediaminetetraacetic acid) or EGTA (ethylene glycol tetraacetic acid), ethyl alcohol, polyethylene glycol, propylene glycol, sodium hydroxide, hydrochloric acid, citric acid, or lactic acid. Antimicrobial agents utilized as carriers may be added to pharmaceutical compositions in multiple-dose containers that include phenols or cresols, mercurials, benzyl alcohol, chlorobutanol, methyl and propyl p-hydroxybenzoic acid esters, thimerosal, benzalkonium chloride and benzethonium chloride. Suitable excipients may include, for example, water, saline, dextrose, glycerol, or ethanol. Suitable non-toxic auxiliary substances may include, for example, wetting or emulsifying agents, pH buffering agents, stabilizers, solubility enhancers, or agents such as sodium acetate, sorbitan monolaurate, triethanolamine oleate, or cyclodextrin.

### Administration, Formulation and Dosage

The pharmaceutical composition of the disclosure may be administered in vivo, to a subject in need thereof, by various routes, including, but not limited to, oral, intravenous, intra-arterial, subcutaneous, parenteral, intranasal, intramuscular, intracranial, intracardiac, intraventricular, intratracheal, buccal, rectal, intraperitoneal, intradermal, topical, transdermal, and intrathecal, or otherwise by implantation or inhalation. The subject compositions may be formulated into preparations in solid, semi-solid, liquid, or gaseous forms; including, but not limited to, tablets, capsules, powders, granules, ointments, solutions, suppositories, enemas, injections, inhalants, and aerosols. The appropriate formulation and route of administration may be selected according to the intended application and therapeutic regimen.

Suitable formulations for enteral administration include hard or soft gelatin capsules, pills, tablets, including coated tablets, elixirs, suspensions, syrups or inhalations and controlled release forms thereof.

Formulations suitable for parenteral administration (e.g., by injection), include aqueous or non-aqueous, isotonic, pyrogen-free, sterile liquids (e.g., solutions, suspensions), in which the active ingredient is dissolved, suspended, or otherwise provided (e.g., in a liposome or other microparticulate). Such liquids may additional contain other pharmaceutically acceptable ingredients, such as anti-oxidants, buffers, preservatives, stabilisers, bacteriostats, suspending agents, thickening agents, and solutes which render the formulation isotonic with the blood (or other relevant bodily fluid) of the intended recipient. Examples of excipients include, for example, water, alcohols, polyols, glycerol, vegetable oils, and the like. Examples of suitable isotonic carriers for use in such formulations include Sodium Chloride Injection, Ringer's Solution, or Lactated Ringer's Injection. Similarly, the particular dosage regimen, including dose, timing and repetition, will depend on the particular individual and that individual's medical history, as well as empirical considerations such as pharmacokinetics (e.g., half-life, clearance rate, etc.).

Frequency of administration may be determined and adjusted over the course of therapy, and is based on reducing the number of proliferative or tumorigenic cells, maintaining the reduction of such neoplastic cells, reducing the proliferation of neoplastic cells, or delaying the development of metastasis. In some embodiments, the dosage administered may be adjusted or attenuated to manage potential side effects and/or toxicity. Alternatively, sustained continuous release formulations of a subject therapeutic composition may be appropriate.

It will be appreciated by one of skill in the art that appropriate dosages can vary from patient to patient. Determining the optimal dosage will generally involve the balancing of the level of therapeutic benefit against any risk or deleterious side effects. The selected dosage level will depend on a variety of factors including, but not limited to, the activity of the particular compound, the route of administration, the time of administration, the rate of excretion of the compound, the duration of the treatment, other drugs, compounds, and/or materials used in combination, the severity of the condition, and the species, sex, age, weight, condition, general health, and prior medical history of the patient. The amount of compound and route of administration will ultimately be at the discretion of the physician, veterinarian, or clinician, although generally the dosage will be selected to achieve local concentrations at the site of action that achieve the desired effect without causing substantial harmful or deleterious side-effects.

In general, the antibody or the antigen binding portion thereof of the disclosure may be administered in various ranges. These include about 5 μg/kg body weight to about 100 mg/kg body weight per dose; about 50 μg/kg body weight to about 5 mg/kg body weight per dose; about 100 μg/kg body weight to about 10 mg/kg body weight per dose. Other ranges include about 100 μg/kg body weight to about 20 mg/kg body weight per dose and about 0.5 mg/kg body weight to about 20 mg/kg body weight per dose. In certain embodiments, the dosage is at least about 100 μg/kg body weight, at least about 250 μg/kg body weight, at least about 750 μg/kg body weight, at least about 3 mg/kg body weight, at least about 5 mg/kg body weight, at least about 10 mg/kg body weight per dose.

In any event, the antibody or the antigen binding portion thereof of the disclosure is preferably administered as needed to a subject in need thereof. Determination of the frequency of administration may be made by persons skilled in the art, such as an attending physician based on considerations of the condition being treated, age of the subject being treated, severity of the condition being treated, general state of health of the subject being treated and the like.

In certain preferred embodiments, the course of treatment involving the antibody or the antigen-binding portion thereof of the instant disclosure will comprise multiple doses of the selected drug product over a period of weeks or months. More specifically, the antibody or the antigen-binding portion thereof of the instant disclosure may be administered once every day, every two days, every four days, every week, every ten days, every two weeks, every three weeks, every month, every six weeks, every two months, every ten weeks or every three months. In this regard, it will be appreciated that the dosages may be altered or the interval may be adjusted based on patient response and clinical practices.

Dosages and regimens may also be determined empirically for the disclosed therapeutic compositions in individuals who have been given one or more administration(s). For example, individuals may be given incremental dosages of a therapeutic composition produced as described herein. In selected embodiments, the dosage may be gradually increased or reduced or attenuated based respectively on empirically determined or observed side effects or toxicity. To assess efficacy of the selected composition, a marker of the specific disease, disorder or condition can be followed as described previously. For cancer, these include direct measurements of tumor size via palpation or visual observation, indirect measurement of tumor size by x-ray or other imaging techniques; an improvement as assessed by direct tumor biopsy and microscopic examination of the tumor sample; the measurement of an indirect tumor marker (e.g., PSA for prostate cancer) or a tumorigenic antigen identified according to the methods described herein, a decrease in pain or paralysis; improved speech, vision, breathing or other disability associated with the tumor; increased appetite; or an increase in quality of life as measured by accepted tests or prolongation of survival. It will be apparent to one of skill in the art that the dosage will vary depending on the individual, the type of neoplastic condition, the stage of neoplastic condition, whether the neoplastic condition has begun to metastasize to other location in the individual, and the past and concurrent treatments being used.

Compatible formulations for parenteral administration (e.g., intravenous injection) will comprise the antibody or antigen-binding portion thereof as disclosed herein in concentrations of from about 10 μg/ml to about 100 mg/ml. In certain selected embodiments, the concentrations of the antibody or the antigen binding portion thereof will comprise 20 μg/ml, 40 μg/ml, 60 μg/ml, 80 μg/ml, 100 μg/ml, 200 μg/ml, 300, μg/ml, 400 μg/ml, 500 μg/ml, 600 μg/ml, 700 μg/ml, 800 μg/ml, 900 μg/ml or 1 mg/ml. In other preferred embodiments, the concentrations of the antibody or the antigen binding portion thereof will comprise 2 mg/ml, 3 mg/ml, 4 mg/ml, 5 mg/ml, 6 mg/ml, 8 mg/ml, 10 mg/ml, 12 mg/ml, 14 mg/ml, 16 mg/ml, 18 mg/ml, 20 mg/ml, 25 mg/ml, 30 mg/ml, 35 mg/ml, 40 mg/ml, 45 mg/ml, 50 mg/ml, 60 mg/ml, 70 mg/ml, 80 mg/ml, 90 mg/ml or 100 mg/ml.

### Applications of the Disclosure

In some aspects, the present disclosure provides a method of treating a disorder in a subject, which comprises administering to the subject (for example, a human) in need of treatment a therapeutically effective amount of the antibody or antigen-binding portion thereof as disclosed herein. For example, the disorder is a cancer.

A variety of cancers where PD-L1 and/or VEGF is implicated, whether malignant or benign and whether primary or secondary, may be treated or prevented with a method provided by the disclosure. The cancers may be solid cancers or hematologic malignancies. In some embodiemnts, the antibodies as disclosed herein may be used for the prevention, amelioration, and treatment of cancers that are related to PD-L1 and/or VEGF, such as colonic cancer and colorectal cancer.

In some other embodiments, the antibodies as disclosed herein may be used for the prevention, amelioration, and treatment of disorders related to angiogenesis.

In some other embodiments, the disorder is an autoimmune disease. Examples of autoimmune diseases that may be treated with the antibody or antigen-binding portion thereof include autoimmune encephalomyelitis, lupus erythematosus, and rheumatoid arthritis. The antibody or the antigen-binding portion thereof may also be used to treat or prevent infectious disease, inflammatory disease (such as allergic asthma) and chronic graft-versus-host disease.

Combined Use with Chemotherapies

The antibody or the antigen-binding portion thereof may be used in combination with an anti-cancer agent, a cytotoxic agent or chemotherapeutic agent.

The term “anti-cancer agent” or “anti-proliferative agent” means any agent that can be used to treat a cell proliferative disorder such as cancer, and includes, but is not limited to, cytotoxic agents, cytostatic agents, anti-angiogenic agents, debulking agents, chemotherapeutic agents, radiotherapy and radiotherapeutic agents, targeted anti-cancer agents, BRMs, therapeutic antibodies, cancer vaccines, cytokines, hormone therapies, radiation therapy and anti-metastatic agents and immunotherapeutic agents. It will be appreciated that, in selected embodiments as discussed above, such anti-cancer agents may comprise conjugates and may be associated with the disclosed site-specific antibodies prior to administration. More specifically, in certain embodiments selected anti-cancer agents will be linked to the unpaired cysteines of the engineered antibodies to provide engineered conjugates as set forth herein. Accordingly, such engineered conjugates are expressly contemplated as being within the scope of the instant disclosure. In other embodiments, the disclosed anti-cancer agents will be given in combination with site-specific conjugates comprising a different therapeutic agent as set forth above.

As used herein the term “cytotoxic agent” means a substance that is toxic to the cells and decreases or inhibits the function of cells and/or causes destruction of cells. In certain embodiments, the substance is a naturally occurring molecule derived from a living organism. Examples of cytotoxic agents include, but are not limited to, small molecule toxins or enzymatically active toxins of bacteria (e.g., Diptheria toxin, Pseudomonas endotoxin and exotoxin, Staphylococcal enterotoxin A), fungal (e.g., α-sarcin, restrictocin), plants (e.g., abrin, ricin, modeccin, viscumin, pokeweed anti-viral protein, saporin, gelonin, momoridin, trichosanthin, barley toxin, Aleurites fordii proteins, dianthin proteins, Phytolacca mericana proteins (PAPI, PAPII, and PAP-S), Momordica charantia inhibitor, curcin, crotin, Saponaria officinalis inhibitor, gelonin, mitegellin, restrictocin, phenomycin, neomycin, and the tricothecenes) or animals, (e.g., cytotoxic Rnases, such as extracellular pancreatic Rnases; Dnase I, including fragments and/or variants thereof).

For the purposes of the instant disclosure a “chemotherapeutic agent” comprises a chemical compound that non-specifically decreases or inhibits the growth, proliferation, and/or survival of cancer cells (e.g., cytotoxic or cytostatic agents). Such chemical agents are often directed to intracellular processes necessary for cell growth or division, and are thus particularly effective against cancerous cells, which generally grow and divide rapidly. For example, vincristine depolymerizes microtubules, and thus inhibits cells from entering mitosis. In general, chemotherapeutic agents can include any chemical agent that inhibits, or is designed to inhibit, a cancerous cell or a cell likely to become cancerous or generate tumorigenic progeny (e.g., TIC). Such agents are often administered, and are often most effective, in combination, e.g., in regimens such as CHOP or FOLFIRI.

Examples of anti-cancer agents that may be used in combination with the antibodies of the present disclosure (either as a component of a site specific conjugate or in an unconjugated state) include, but are not limited to, alkylating agents, alkyl sulfonates, aziridines, ethylenimines and methylamelamines, acetogenins, a camptothecin, bryostatin, callystatin, CC-1065, cryptophycins, dolastatin, duocarmycin, eleutherobin, pancratistatin, a sarcodictyin, spongistatin, nitrogen mustards, antibiotics, enediyne antibiotics, dynemicin, bisphosphonates, esperamicin, chromoprotein enediyne antiobiotic chromophores, aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, ADRIAMYCIN® doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites, erlotinib, vemurafenib, crizotinib,sorafenib, ibrutinib, enzalutamide, folic acid analogues, purine analogs, androgens, anti-adrenals, folic acid replenisher such as frolinic acid, aceglatone, aldophosphamide glycoside, aminolevulinic acid, eniluracil, amsacrine, bestrabucil, bisantrene, edatraxate, defofamine, demecolcine, diaziquone, elfornithine, elliptinium acetate, an epothilone, etoglucid, gallium nitrate, hydroxyurea, lentinan, lonidainine, maytansinoids, mitoguazone, mitoxantrone, mopidanmol, nitraerine, pentostatin, phenamet, pirarubicin, losoxantrone, podophyllinic acid, 2-ethylhydrazide, procarbazine, PSK® polysaccharide complex (JHS Natural Products, Eugene, OR), razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, chloranbucil; GEMZAR® gemcitabine; 6-thioguanine; mercaptopurine; methotrexate; platinum analogs, vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; irinotecan (Camptosar, CPT-11), topoisomerase inhibitor RFS 2000; difluorometlhylornithine; retinoids; capecitabine; combretastatin; leucovorin; oxaliplatin; inhibitors of PKC-alpha, Raf, H-Ras, EGFR and VEGF-A that reduce cell proliferation and pharmaceutically acceptable salts, acids or derivatives of any of the above. Also included in this definition are anti-hormonal agents that act to regulate or inhibit hormone action on tumors such as anti-estrogens and selective estrogen receptor modulators, aromatase inhibitors that inhibit the enzyme aromatase, which regulates estrogen production in the adrenal glands, and anti-androgens; as well as troxacitabine (a 1,3-dioxolane nucleoside cytosine analog); antisense oligonucleotides, ribozymes such as a VEGF expression inhibitor and a HER2 expression inhibitor; vaccines, PROLEUKIN® rIL-2; LURTOTECAN® topoisomerase 1 inhibitor; ABARELIX® rmRH; Vinorelbine and Esperamicins and pharmaceutically acceptable salts, acids or derivatives of any of the above.

Combined Use with Radiotherapies

The present disclosure also provides for the combination of the antibody or the antigen-binding portion thereof with radiotherapy (i.e., any mechanism for inducing DNA damage locally within tumor cells such as gamma-irradiation, X-rays, UV-irradiation, microwaves, electronic emissions and the like). Combination therapy using the directed delivery of radioisotopes to tumor cells is also contemplated, and the disclosed antibodies may be used in connection with a targeted anti-cancer agent or other targeting means. Typically, radiation therapy is administered in pulses over a period of time from about 1 to about 2 weeks. The radiation therapy may be administered to subjects having head and neck cancer for about 6 to 7 weeks. Optionally, the radiation therapy may be administered as a single dose or as multiple, sequential doses.

### Pharmaceutical Packs and Kits

Pharmaceutical packs and kits comprising one or more containers, comprising one or more doses of the antibody or the antigen-binding portion thereof are also provided. In certain embodiments, a unit dosage is provided wherein the unit dosage contains a predetermined amount of a composition comprising, for example, the antibody or the antigen-binding portion thereof, with or without one or more additional agents. For other embodiments, such a unit dosage is supplied in single-use prefilled syringe for injection. In still other embodiments, the composition contained in the unit dosage may comprise saline, sucrose, or the like; a buffer, such as phosphate, or the like; and/or be formulated within a stable and effective pH range. Alternatively, in certain embodiments, the composition may be provided as a lyophilized powder that may be reconstituted upon addition of an appropriate liquid, for example, sterile water or saline solution. In certain preferred embodiments, the composition comprises one or more substances that inhibit protein aggregation, including, but not limited to, sucrose and arginine. Any label on, or associated with, the container(s) indicates that the enclosed composition is used for treating the neoplastic disease condition of choice.

The present disclosure also provides kits for producing single-dose or multi-dose administration units of antibodies and, optionally, one or more anti-cancer agents. The kit comprises a container and a label or package insert on or associated with the container. Suitable containers include, for example, bottles, vials, syringes, etc. The containers may be formed from a variety of materials such as glass or plastic and contain a pharmaceutically effective amount of the disclosed antibodies, either in a conjugated or unconjugated form. In other preferred embodiments, the container(s) comprise a sterile access port (for example the container may be an intravenous solution bag or a vial having a stopper pierceable by a hypodermic injection needle). Such kits will generally contain in a suitable container a pharmaceutically acceptable formulation of the antibodies and, optionally, one or more anti-cancer agents in the same or different containers. The kits may also contain other pharmaceutically acceptable formulations, either for diagnosis or combined therapy. For example, in addition to the antibody or the antigen-binding portion thereof of the disclosure such kits may contain any one or more of a range of anti-cancer agents such as chemotherapeutic or radiotherapeutic drugs; anti-angiogenic agents; anti-metastatic agents; targeted anti-cancer agents; cytotoxic agents; and/or other anti-cancer agents.

More specifically the kits may have a single container that contains the disclosed the antibody or the antigen-binding portion thereof, with or without additional components, or they may have distinct containers for each desired agent. Where combined therapeutics are provided for conjugation, a single solution may be pre-mixed, either in a molar equivalent combination, or with one component in excess of the other. Alternatively, the antibodies and any optional anti-cancer agent of the kit may be maintained separately within distinct containers prior to administration to a patient. The kits may also comprise a second/third container means for containing a sterile, pharmaceutically acceptable buffer or other diluent such as bacteriostatic water for injection (BWFI), phosphate-buffered saline (PBS), Ringer's solution and dextrose solution.

When the components of the kit are provided in one or more liquid solutions, the liquid solution is preferably an aqueous solution, with a sterile aqueous or saline solution being particularly preferred. However, the components of the kit may be provided as dried powder(s). When reagents or components are provided as a dry powder, the powder can be reconstituted by the addition of a suitable solvent. It is envisioned that the solvent may also be provided in another container.

As indicated briefly above the kits may also contain a means by which to administer the antibody or the antigen-binding portion thereof and any optional components to a patient, e.g., one or more needles, I.V. bags or syringes, or even an eye dropper, pipette, or other such like apparatus, from which the formulation may be injected or introduced into the animal or applied to a diseased area of the body. The kits of the present disclosure will also typically include a means for containing the vials, or such like, and other component in close confinement for commercial sale, such as, e.g., injection or blow-molded plastic containers into which the desired vials and other apparatus are placed and retained.

### Sequence Listing Summary

Appended to the instant application is a sequence list comprising a number of amino acid sequences. The following Table A provides a summary of the included sequences.

One illustrative antibody as disclosed herein, which is an anti-VEGF/anti-PD-L1 bispecific antibody, is designated as W3253-U9T2.G17-1.uIgG1V320 (abbreviated as “W3253”).

TABLE A

| SEQ ID | |
|---|---|
| NO. | Description |
| 1 | Amino acid sequence of HCDR1 of PD-L1 binding moiety |
| 2 | Amino acid sequence of HCDR2 of PD-L1 binding moiety |
| 3 | Amino acid sequence of HCDR3 of PD-L1 binding moiety |
| 4 | Amino acid sequence of LCDR1 of PD-L1 binding moiety |
| 5 | Amino acid sequence of LCDR2 of PD-L1 binding moiety |
| 6 | Amino acid sequence of LCDR3 of PD-L1 binding moiety |
| 7 | Amino acid sequence of HCDR1 of VEGF binding moiety |
| 8 | Amino acid sequence of HCDR2 of VEGF binding moiety |
| 9 | Amino acid sequence of HCDR3 of VEGF binding moiety |
| 10 | Amino acid sequence of LCDR1 of VEGF binding moiety |
| 11 | Amino acid sequence of LCDR2 of VEGF binding moiety |
| 12 | Amino acid sequence of LCDR3 of VEGF binding moiety |
| 13 | Amino acid sequence of VH of PD-L1 binding moiety |
| 14 | Amino acid sequence of VL of PD-L1 binding moiety |
| 15 | Amino acid sequence of VH of VEGF binding moiety |
| 16 | Amino acid sequence of VL of VEGF binding moiety |
| 17 | Amino acid sequence of the heavy chain of W3253 antibody |
| 18 | Amino acid sequence of the light chain of W3253 antibody |
| 19 | Nucleiotide sequence encoding heavy chain of W3253 antibody |
| 20 | Nucleiotide sequence encoding light chain of W3253 antibody |

### EXAMPLES

The present disclosure, thus generally described, will be understood more readily by reference to the following Examples, which are provided by way of illustration and are not intended to be limiting of the present disclosure. The Examples are not intended to represent that the experiments below are all or the only experiments performed.

### Example 1

### Preparation of Materials, Benchmark Antibodies and Cell Lines

### 1.1 Preparation of Materials

Information on the commercially available materials used in the examples are provided in Table 1.

TABLE 1

| Materials | Vendor | Cat. |
|---|---|---|
| Expi293 Expression System | Thermo Fisher | A14635 |
| | Scientific | |
| Protein A column | GE healthcare | 17-5438-02 |
| Ni column | GE healthcare | 17-5247-01 |
| HPLC-SEC | TOSOH | 0008541 |
| NuPAGE4%-12% Bis-Tris Gel | Invitrogen | NP0322BOX |
| Human VEGF | Sino Biological | 11066-HNAB |
| Human VEGF, Biotin labeled | ACRO Biosystems | VE5-H8210 |
| Human VEGFR1, hFc tag | Sino Biological | 10136-H02H |
| Human VEGFR2, hFc tag | Sino Biological | 10012-H02H1 |
| Human PD-L1, His tag | Sino Biological/ | 10084-H08H |
| | In house | |
| Cynomolgus PD-L1, His tag | Sino Biological/ | 90251-C08H |
| | In house | |
| Human PD-L1, mFc tag | In house | |
| Human PD-1, mFc tag | In house | |
| Goat-anti-human IgG Fc-HRP | Bethyl | A80-304P |
| antibody | | |
| RPMI 1640 | Gibco | 22400-089 |
| Goat x-human IgG Fc FITC | Bethyl | A80-304F |
| Conjugated | | |
| Recombinant human IL-2 standard | R&D | 202-IL-050 |
| Recombinant human IFN γ | PeproTech | 300-02 |
| standard | | |
| anti-hIL-2 purified Mouse | R&D | MAB602 |
| Monoclonal IgG2A Clone 5355 | | |
| anti-hIL-2 Biotinylated Goat IgG | R&D | BAF202 |
| Human IFNγ Mab clone 2G1 | Thermo prod | M700A |
| Human IFNγ Mab Biotin-Labeled | Thermo prod | M701B |
| Streptavidin HRP | Invitrogen | SNN1004 |
| Fetal bovine serum (FBS) | ExCell Bio | FND500 |
| penicillin-streptomycin (PS) | Invitrogen | SV30010 |

### 1.2 Antigen Preparation

DNA sequences encoding the sequence of human VEGF (Uniport No.: P15692), the extracellular domain sequence of human PD-L1 (Uniport No.: Q9NZQ7), cynomolgus monkey PD-1 (Uniport No.: Q15116), and human PD-1 (Uniport No.: Q15116) were synthesized in Sangon Biothech (Shanghai, China), and then subcloned into modified pcDNA3.3 expression vectors with different tag (such as 6xhis, human Fc, or mouse Fc) at C-terminus.

Expi293 cells (Thermo Fisher Scientific, A14527) were transfected with the purified expression vector. Cells were cultured for 5 days and supernatant was collected for protein purification using Ni-NTA column (GE Healthcare, 175248), Protein A column (GE Healthcare, 175438) or Protein G column (GE Healthcare, 170618). The obtained human VEGF, human PD-L1 and human PD-1 were QC′ed by SDS-PAGE and SEC, and then stored at −80° C.

### 1.3 Production of Benchmark Antibodies (BMK Abs)

DNA sequences encoding the variable regions of bevacizumab (i.e. Avastin, sequence from Drug Bank, Drug Bank No.: DB00112) and atezolizumab (anti-PD-L1 antibody developed by Roche) were synthesized in Sangon Biothech (Shanghai, China) or Genewiz (Suzhou, China), and then subcloned into modified pcDNA3.3 expression vectors with constant region of human IgG1.

The plasmids encoding heavy chain and light chain were co-transfected into Expi293 cells. Cells were cultured for 5 days and supernatant was collected for protein purification using Protein A column (GE Healthcare, 175438). The obtained antibodies were analyzed by SDS-PAGE and SEC-HPLC, and then stored at −80° C.

### 1.4 Establishment of Stable Cell Lines/Cell Pool

### PD-L1-Expressing Cell Lines

Human PD-L1 high expression stable cell line (WBP315.CHO-K1.hPro1.C11) and cynomolgus monkey PD-L1 high expression stable cell line (WBP315.293F.cPro1.2A) were obtained by limiting dilution.

Briefly, the genes of human PD-L1 or cynomolgus monkey PD-L1 were inserted into expression vector pcDNA 3.3 respectively. The plasmids were then transfected to CHO-K1/293F cells, respectively. 6-8 hours after transfection, cells were washed with PBS and feeded with 3 ml of fresh non-selective media in 6-well plate. Cells were harvested with trypsin 24-48 hours post-transfection and plated to T75 flask in selective media (F12-K, 10% FBS, 10 μg/ml Blasticidin). High expression stable cell line was obtained by limiting dilution, and expression level was determined by FACS.

### Immortal Cell Line

Human Umbilical Vein Endothelial Cell (HUVEC) was purchased from ScienceCell (Cat: 8000) and cultured in endothelial cell medium (ECM, ScienceCell, Cat: 1001) containing basic medium, 5% FBS, 1% endothelial cell growth factor (ECGS, ScienceCell, 1052). The cells were cultured in an incubator with 37° C. and 5% CO₂. For long term storage, the cells were frozen in complete growth medium supplemented with 5% (v/v) DMSO and stored in liquid nitrogen vapor phase.

### Example 2

### Generation of PD-L1/VEGF Bispecific Antibodies

For BsAb construction, anti-PD-L1 fully human monoclonal antibody was generated in house by OMT rat immunization and hybridoma technology, and the obtained anti-PD-L1 monoclonal antibody was identified and named as W3155-r1.14.4. The heavy chain and light chain variable regions were as shown in SEQ ID NO: 13 and 14, respectively. The variable regions of W3155-r1.14.4 and bevacizumab were used for constructing the bispecific antibody.

The heavy chain and light chain variable regions of W3155-r1.14.4 were combined in one chain to form an anti-PD-L1 scFv. DNA sequence encoding anti-PD-L1 scFv (VH-(G4S)4-VL) was added to the N-terminal of heavy chain of bevacizumab with (G4S)2 linker, in which Fc region contained LALA mutation to abrogate Fc effector function. The light chain was same as bevacizumab. The constructed BsAb was named as W3253-U9T2.G17-1.uIgG1V320 (suffix “V320” means IgG1 Fc region comprises a L234A/L235A substitution) or W3253. Then DNA sequences encoding both chains were cloned into modified pcDNA3.3 expression vectors.

Heavy chain and light chain expression plasmids were co-transfected into Expi293 cells using Expi293 expression system kit (ThermoFisher-A14635) according to the manufacturer's instructions. 5 days after transfection, the supernatants were collected and used for protein purification using Protein A column and CEX column. Antibody concentration was measured by NanoDrop. The purity of proteins was evaluated by SDS-PAGE and HPLC-SEC (FIGS. 2 and 3). The specific sequences of W3253-U9T2.G17-1.uIgG1V320 antibody are listed in Tables 2-4 below.

TABLE 2

| | | | |
|---|---|---|---|
| Anti-VEGF | HCDR1 | HCDR2 | HCDR3 |
| antibody | SEQ ID | SEQ ID | SEQ ID |
| (bevacizumab) | NO: 7 | NO: 8 | NO: 9 |
| | GYTFT | WINTYTGE | YPHYYGS |
| | NYGMN | PTYAADFKR | SHWYFDV |
| | LCDR1 | LCDR2 | LCDR3 |
| | SEQ ID | SEQ ID | SEQ ID |
| | NO: 10 | NO: 11 | NO: 12 |
| | SASQDI | YFTS | QQYST |
| | SNYLN | SLHS | VPWT |
| Anti-PD-L1 | HCDR1 | HCDR2 | HCDR3 |
| ScFv | SEQ ID | SEQ ID | SEQ ID |
| (W3155- | NO: 1 | NO: 2 | NO: 3 |
| r1.14.4) | GFTFS | GISGSGGF | PPRGYNY |
| | SYAMS | TYYADSVKG | GPFDY |
| | LCDR1 | LCDR2 | LCDR3 |
| | SEQ ID | SEQ ID | SEQ ID |
| | NO: 4 | NO: 5 | NO: 6 |
| | GGNNIG | DDSD | QVWDSS |
| | SKSVH | RPS | SDHVV |

TABLE 3
Amino acid sequence of VH of VEGF binding
moiety:
EVQLVESGGGLVQPGGSLRLSCAASGYTFTNYGMNWVRQAPGKGL
EWVGWINTYTGEPTYAADFKRRFTFSLDTSKSTAYLQMNSLRAED
TAVYYCAKYPHYYGSSHWYFDVWGQGTLVTVSS
(SEQ ID NO: 15)
Amino acid sequence of VL of VEGF binding
moiety:
DIQMTQSPSSLSASVGDRVTITCSASQDISNYLNWYQQKPGKAPK
VLIYFTSSLHSGVPSRFSGSGSGTDFTLTISSLQPEDFATYYCQQ
YSTVPWTFGQGTKVEIK
(SEQ ID NO: 16)
Amino acid sequence of VH of PD-L1 binding
moiety:
EVQLLESGGGLVQPGGSLRLSCAASGFTESSYAMSWVRQAPGKGL
EWVSGISGSGGFTYYADSVKGRFTISRDNSKNTLYLQMNSLRAED
TAVYYCAKPPRGYNYGPFDYWGQGTLVTVSS
(SEQ ID NO: 13)
Amino acid sequence of VL of PD-L1 binding
moiety:
SYVLTQPPSVSVAPGQTARITCGGNNIGSKSVHWYQQKPGQAPVL
VVYDDSDRPSGIPERFSGSNSGNTATLTISRVEAGDEADYYCQVW
DSSSDHVVFGGGTKLTVL
(SEQ ID NO: 14)

TABLE 4
Amino acid sequence of the heavy chain
of W3253-U9T2.G17-1.uIgG1V320:
EVQLLESGGGLVQPGGSLRLSCAASGFTFSSYAMSWVRQAPGKGL
EWVSGISGSGGFTYYADSVKGRFTISRDNSKNTLYLQMNSLRAED
TAVYYCAKPPRGYNYGPFDYWGQGTLVTVSSGGGGSGGGGSGGGG
SGGGGSSYVLTQPPSVSVAPGQTARITCGGNNIGSKSVHWYQQKP
GQAPVLVVYDDSDRPSGIPERFSGSNSGNTATLTISRVEAGDEAD
YYCQVWDSSSDHVVFGGGTKLTVLGGGGSGGGGSEVQLVESGGGL
VQPGGSLRLSCAASGYTFTNYGMNWVRQAPGKGLEWVGWINTYTG
EPTYAADFKRRFTFSLDTSKSTAYLQMNSLRAEDTAVYYCAKYPH
YYGSSHWYFDVWGQGTLVTVSSASTKGPSVFPLAPSSKSTSGGTA
ALGCLVKDYFPEPVTVSWNSGALTSGVHTFPAVLQSSGLYSLSSV
VTVPSSSLGTQTYICNVNHKPSNTKVDKRVEPKSCDKTHTCPPCP
APEAAGGPSVFLFPPKPKDTLMISRTPEVTCVVVDVSHEDPEVKF
NWYVDGVEVHNAKTKPREEQYNSTYRVVSVLTVLHQDWLNGKEYK
CKVSNKALPAPIEKTISKAKGQPREPQVYTLPPSREEMTKNQVSL
TCLVKGFYPSDIAVEWESNGQPENNYKTTPPVLDSDGSFFLYSKL
TVDKSRWQQGNVFSCSVMHEALHNHYTQKSLSLSPG
(SEQ ID NO: 17)
Amino acid sequence of the light chain
of W3253-U9T2.G17-1.uIgG1V320:
DIQMTQSPSSLSASVGDRVTITCSASQDISNYLNWYQQKPGKAPK
VLIYFTSSLHSGVPSRFSGSGSGTDFTLTISSLQPEDFATYYCQQ
YSTVPWTFGQGTKVEIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSENRGEC
(SEQ ID NO: 18)

### Example 3

### In Vitro Characterization of the Bispecific Antibodies

### 3.1 Human/Cynomolgus Monkey VEGF-Binding (ELISA)

The amino acid sequence of cynomolgus monkey VEGF is the same with human VEGF. For ELISA binding, non-tissue culture treated flat-bottom 96-well plates (Nunc MaxiSorp, ThermoFisher) were pre-coated with 0.25 μg/ml Sino Biological human VEGF protein W325-hPro1 (Sino) overnight at 4° C. After 2% BSA blocking, 100 μL 4-fold titrated antibodies from 200 nM to 0.000190735 nM were pipetted into each well and incubated for 2 hours at ambient temperature. Following removal of the unbound substances, 100 μL 1:5000 diluted HRP-labeled Goat anti-human IgG (Bethyl A80-304P) were added to the wells and incubated for 1 hour. The color was developed by dispensing 100 μL TMB substrate, and then stopped by 100 μL 2 M HCl. The absorbance was read at 450 nm and 540 nm using a Microplate Spectrophotometer (SpectraMax® M5^{e}).

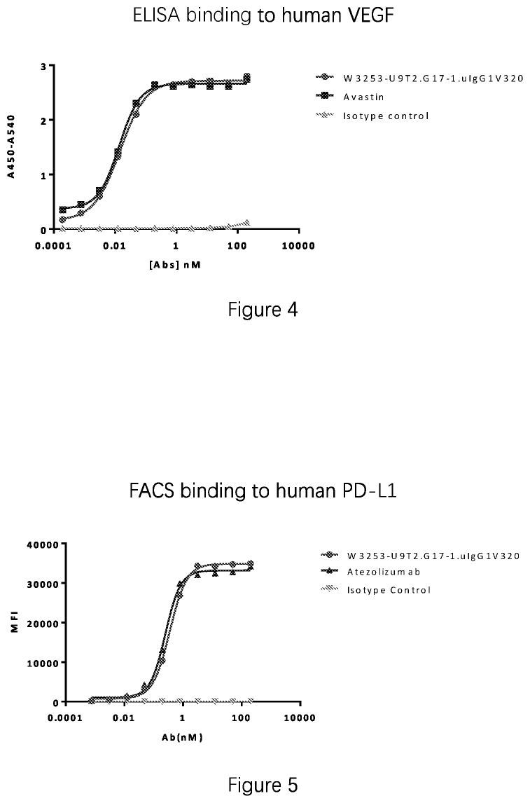

W3253-U9T2.G17-1.uIgG1V320 shows comparable binding ability to its parental antibody Avastin on human VEGF with EC₅₀ of 0.01 nM (FIG. 4).

### 3.2 Human PD-L1 Binding (FACS)

For FACS binding, engineered human PD-L1 expressing cells W315-CHOK1.hPro1.C11 were seeded at 1×10⁵ cells/well in U-bottom 96-well plates (COSTAR 3799). 4-Fold titrated Abs with 1% BSA DPBS from 200 nM to 0.000762939 nM were added to the cells. Plates were incubated at 4° C. for 1 hour. After wash, 100 μL 1:150 diluted PE-labeled goat anti-human antibody (Jackson 109-115-098) was added to each well and the plates were incubated at 4° C. for half an hour. The binding of the antibodies onto the cells was tested by flow cytometry and the mean fluorescence intensity (MFI) was analyzed by FlowJo.

W3253-U9T2.G17-1.uIgG1V320 also shows comparable binding ability to Atezolizumab on human PD-L1 with EC₅₀ of 0.128 nM (FIG. 5).

### 3.3 Human VEGF/Human PD-L1 Dual Binding (ELISA)

In order to test whether the bispecific antibodies could bind to both human PD-L1 and VEGF, an ELISA assay was developed as below. A 96-well ELISA plate (Nunc MaxiSorp, ThermoFisher) was coated overnight at 4° C. with 1 μg/ml antigen-1 (hVEGF.his, W325-hPro1.his (in house) in carbonate-bicarbonate buffer. After a 1 hour blocking step with casein buffer, serial dilutions of the different PD-L1×VEGF bispecific antibodies (5-fold dilution, from 100 nM to 0.00128 nM) in casein buffer were incubated on the plates for 1 hour at room temperature. Following the incubation, plates were washed three times with 300 μL per well of PBS containing 0.5% (v/v) Tween 20. 1 μg/ml antigen-2 (hPD-L1-ECD.mFc, W315-hPro1.ECD.mFc (in house) was added to plates and incubation 1 hour. After washing the plates three times, 100 μL 1:5000 diluted HRP-labeled Goat anti mouse IgG (Bethyl A90-231P) was added and incubated on the plates for 1 hour at room temperature. After washing six times with 300 μL per well of PBS containing 0.5% (v/v) Tween 20, 100 μL TMB substrate was added for the detection pre well. The reaction was stopped after approximate 5 minutes through the addition of 100 μL per well of 2 M HCl. The absorbance of the wells was measured at 450 nm and 540 nm with a multiwall plate reader (SpectraMax® M5^{e}).

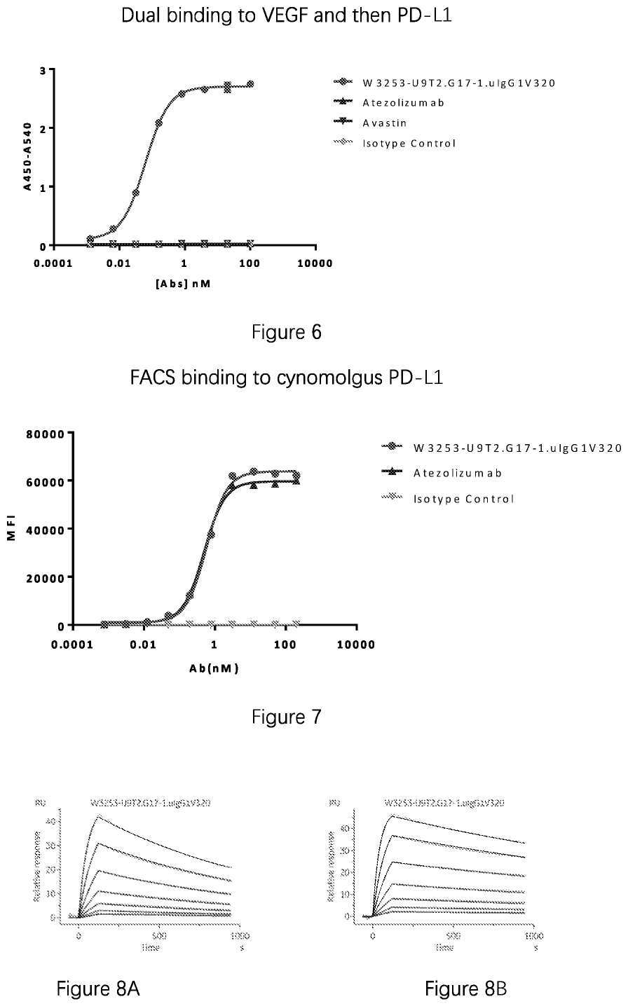

Dual binding activity of W3253-U9T2.G17-1.uIgG1V320 to human VEGF and human PD-L1 were measured by ELISA-based binding assays. The results show that the binding of W3253-U9T2.G17-1.uIgG1V320 didn't affect the subsequent binding of PD-L1 (FIG. 6).

### 3.4 Cynomolgus Monkey PD-L1 Binding (FACS)

For FACS binding, engineered cyno PD-L1 expressing cells W315-293F.cPro1.2A2 (in house) were seeded at 1×10⁵ cells/well in U-bottom 96-well plates (COSTAR 3799). 4-Fold titrated Abs with 1% BSA DPBS from 200 nM to 0.000762939 nM were added to the cells. Plates were incubated at 4° C. for 1 hour. After wash, 100 μL 1:150 diluted PE-labeled goat anti-human antibody (Jackson 109-115-098) was added to each well and the plates were incubated at 4° C. for half an hour. The binding of the antibodies onto the cells was tested by flow cytometry and the mean fluorescence intensity (MFI) was analyzed by FlowJo.

The amino acid of cynomolgus monkey VEGF is the same with human VEGF, therefore, W3253-U9T2.G17-1.uIgG1V320 also has binding activity to cynomolgus VEGF. W3253-U9T2.G17-1.uIgG1V320 shows comparable binding ability to the Atezolizumab on cynomolgus PD-L1 with EC₅₀ of 0.59 nM (FIG. 7).

### 3.5 Affinity of Binding to VEGF and PD-L1 (SPR)

Antibodies binding affinity to the antigen were detected by SPR assay using Biacore 8K. Abs were captured on an anti-human IgG Fc antibody immobilized CM5 sensor chip (GE). The antigen at different concentrations were injected over the sensor chip at a flow rate of 30 μL/min for an association phase, followed by a dissociation phase. The chip was regenerated by 10 mM glycine (pH 1.5) after each binding cycle. The sensorgrams of blank surface and buffer channel were subtracted from the test sensorgrams. The experimental data was fitted by 1:1 model using Langmiur analysis.

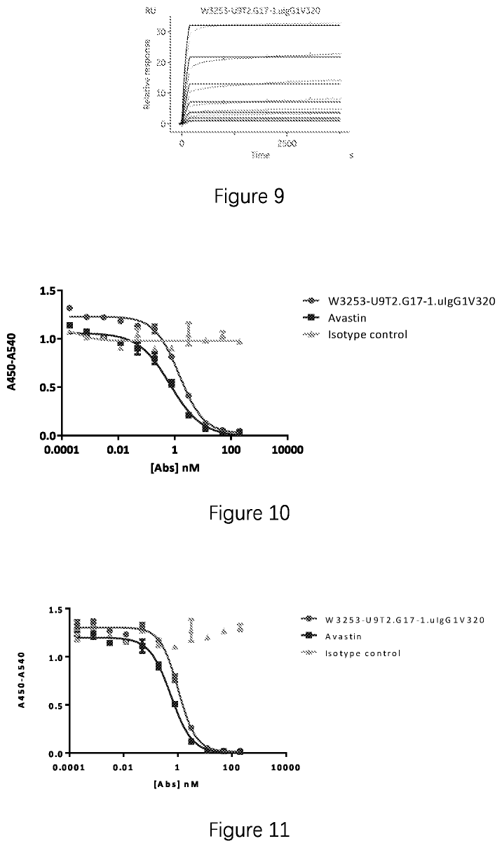

The affinity constant (K_{D}) of W3253-U9T2.G17-1.uIgG1V320 was measured based on SPR technology. The on-rate constant (ka) and off-rate constant (kd) are measured in the meantime. Final data of each interaction was deducted from reference channel and buffer channel data. The experimental data was analysed as shown in FIG. 8A, FIG. 8B and FIG. 9. The Kinetic affinity results of antibodies were listed in Table 5.

TABLE 5

| Analyte | Ligand | ka1 (1/Ms) | kd1 (1/s) | KD1 (M) |
|---|---|---|---|---|
| human PD-L1 (W315- | W3253-U9T2.G17-1.uIgG1V320 | 1.81E+06 | 8.57E−04 | 4.75E−10 |
| hPro1.ECD.His) | Atezolizumab | 1.00E+06 | 9.06E−05 | 9.04E−11 |
| cyno PD-L1 (W315- | W3253-U9T2.G17-1.uIgG1V320 | 2.45E+06 | 3.80E−04 | 1.55E−10 |
| cynoPro1.ECD.His(Sino)) | Atezolizumab | 9.49E+05 | 4.65E−03 | 4.90E−09 |
| human VEGF (W325- | W3253-U9T2.G17-1.uIgG1V320 | 4.26E+05 | <1.00E−05* | <2.35E−11 |
| hPro1(Sino)) | Avastin | 4.81E+05 | <1.00E−05* | <2.08E−11 |

### 3.6 Human VEGFR1 and VEGFR2 Competition Assays

In order to test whether the bispecific antibodies could block human VEGFR1 and VEGFR2 binding to human VEGF protein, ligand competition assays were performed as follows.

In an ELISA, flat-bottom 96-well plates (Nunc MaxiSorp, ThermoFisher) were pre-coated with 0.5 μg/ml W325-hpro1R1.ECD.hFc (SB) or 2 μg/ml W325-hpro1R2.ECD.hFc (SB) overnight at 4° C. After casein buffer blocking, 100 μL 2-fold titrated Abs from 200 nM to 0.000190735 nM coupled with 0.02 μg/ml human VEGF protein W325-hPro1.his.biotin were pipetted into each well and incubated for 2 hours at ambient temperature. Following the incubation, plates were washed 3 times with 300 μL per well of PBS containing 0.5% (v/v) Tween 20. 100 μL 1:10000 diluted Streptavidin-HRP (Lifetechnologies #SNN1004) was added to plate pre well and incubation 1 hour. After wash 6 times, the color was developed by dispensing 100 μL of TMB substrate, and then stopped by 100 μL of 2 M HCl. The absorbance was read at 450 nm and 540 nm using a Microplate Spectrophotometer (SpectraMax® M5^{e}).

W3253-U9T2.G17-1.uIgG1V320 shows better competition ability than the parental antibodies against hVEGFR1 binding to VEGF with IC₅₀ of 1.47 nM (FIG. 10), and shows better competition ability than the parental antibodies against hVEGFR2 binding to VEGF with IC₅₀ of 1.04 nM (FIG. 11).

### 3.7 Human PD-1 Competition Assays

In order to test whether the bispecific antibodies could block PD-1 protein binding to PD-L1 expressing cells, the following competition assay was performed.

For blocking human PD-1 binding to human PD-L1 by FACS, engineered human PD-L1 expressing cells W315-CHOK1.hPro1.C11 were seeded at 1×10⁵ cells/well in U-bottom 96-well plates (COSTAR 3799). 4-Fold titrated Abs from 200 nM to 0.000762939 nM coupled with 5 ug/ml in house human PD-1 protein W305-hPro1.ECD.mFc were added to the cells. Plates were incubated at 4° C. for 1 hour. After washing, 100 μL 1:150 diluted PE-labeled Goat anti-mouse antibody (abcam 98742) was added to each well and the plates were incubated at 4° C. for half an hour. The competition binding of antibodies to the cells was tested by flow cytometry and the mean fluorescence intensity (MFI) was analyzed by FlowJo.

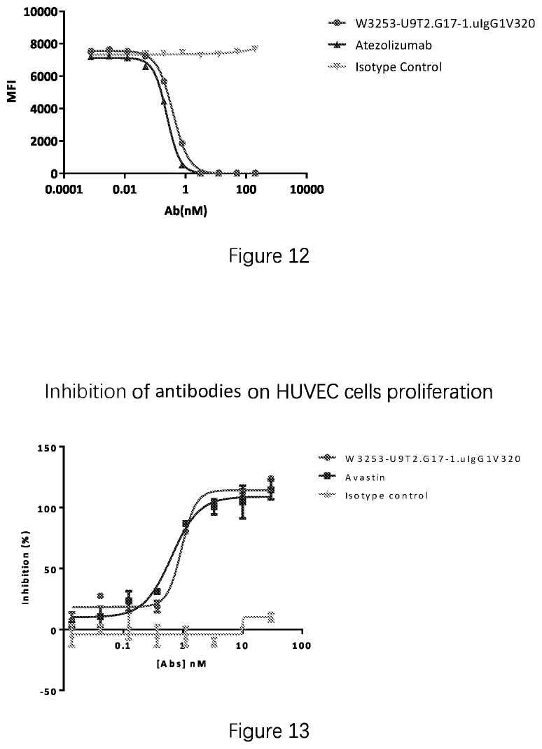

W3253-U9T2.G17-1.uIgG1V320 shows comparable competition ability with Atezolizumab in blocking the binding between human PD-1 and PD-L1 with IC₅₀ of 0.39 nM (FIG. 12).

### 3.8 HUVEC Cell Proliferation Assay

The biological activity of W3253-U9T2.G17-1.uIgG1V320 in VEGF-induced HUVEC proliferation was assessed. HUVEC cells were routinely cultured in ECM+5% FBS+1% ECGS. Sub-confluent cells were harvested by trypsin, diluted to 1×10⁵ cells/mL with ECM+1% FBS+0.05% ECGS. Cells were plated in 96-well clear bottom black plates (Greiner, 655090) at a density of 4000 cells/well. Serial diluted antibodies were added, together with 50 ng/mL of human VEGF (WBP325-hPro1, Sino Biological, 11066-HNAB). The plates were returned to the incubator for 5 days before assessing cell viability using CellTiter Glo (Promega, G7573). Wells with no ligand addition served as control for ligand stimulated cell growth. The effect of the tested antibody on inhibiting ligand stimulated cell growth was calculated by comparing the luminescence values with or without antibody addition (ligand only) after subtracting the background (no ligand) luminescence. Four-parameter non-linear regression analysis was used to obtain proliferation inhibition IC₅₀ values using GraphPad Prism 5 software.

W3253-U9T2.G17-1.uIgG1V320 effectively blocked VEGF induced HUVEC proliferation in a concentration-dependent manner with IC₅₀ of 0.95 nM and maximum inhibition rate of 114.2% (FIG. 13).

### 3.9 Reporter Gene Assay

To test whether WBP3253 lead antibody can functionally counteract the role of PD-L1 in regulating T cell response, Jurakt cells with full length PD-1-expression and integrated with NFAT-RE-Luc2p (effect cells) and CHO-K1-PD-L1 cells with anti-CD3 (OTK3) ScFv Ab(target cells) were seeded at 2×10⁴ cells/50 μL and 4×10⁴ cells/50 μL respectively. And then co-culture with 50 μL WBP3253 Abs (4-fold dilution, form 33.5 nM to 0.002045 nM) at 37° C. for 6 hours. Following the incubation, 50 μL one-glo luciferase substrate was added to each well for developing. The fluorescence intensity of the wells was measured by EnVision (NO. 798104).

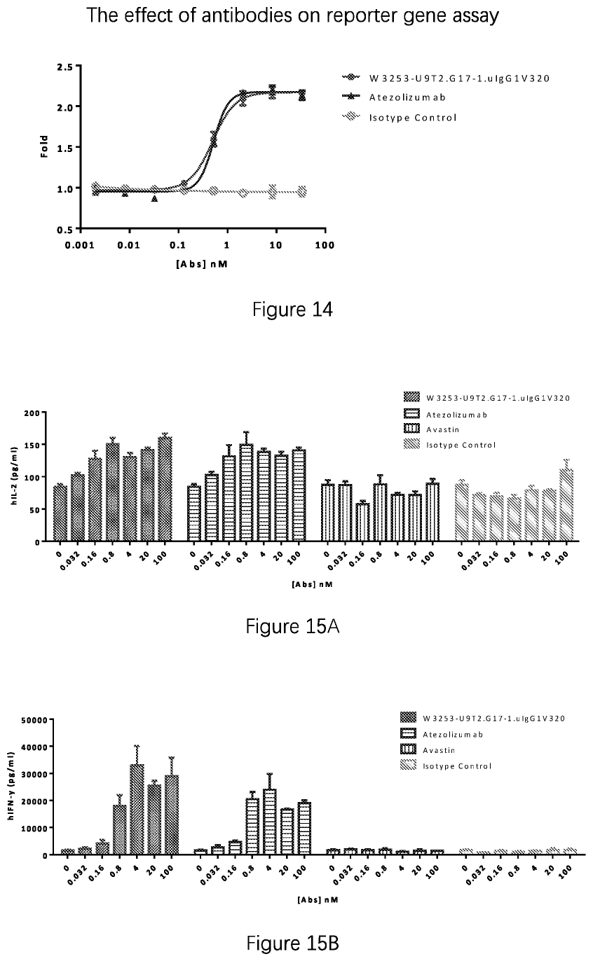

W3253-U9T2.G17-1.uIgG1V320 shows functionality in PD-L1 report gene assay (FIG. 14).

### 3.10 Mixed Lymphocyte Reaction (MLR) Assays

MLR was used to test the agonistic effect of PD-L1 antibodies on cytokine, human IFN-γ secretion and proliferation of activated human CD4⁺ T cells.

Human peripheral blood mononuclear cells (PBMCs) were freshly isolated from healthy donors using Ficoll-Paque PLUS gradient centrifugation. Monocytes were isolated using Human Monocyte Enrichment Kit (Miltenyi Biotec-130-050-201) according to the manufacturer's instructions. Cell concentration was adjusted to 2×10⁶ cells/ml in complete RPMI-1640 medium (Gibco-22400089) supplemented with recombinant human GM-CSF at 800 U/ml and IL-4 at 50 μg/ml at 2.5 ml/well in 6-well plate. Cells were cultured for 5 to 7 days to differentiate into dendritic cells (DC). Cytokines were replenished every 2-3 days by replacing half of the media with fresh media supplemented with cytokines. Human CD4⁺ T cells were isolated using Human CD4⁺ T Cell Enrichment kit according to the manufacturer's protocol.

MLR was set up in 96-well round bottom plates (Nunc, 163320) using complete RPMI-1640 medium. CD4⁺ T cells, various concentrations of antibodies, and immature DCs were added to the plates. The plates were incubated at 37° C., 5% CO₂. IFN-γ production was determined at day 5.

Human IFN-γ was measured by enzyme-linked immunosorbent assay (ELISA) using matched antibody pairs. Recombinant human IFN-γ (PeproTech, 300-02) was used as standards, respectively. The plates were pre-coated with capture antibody specific for human IFN-γ (Pierce, M700A). After blocking, standards or samples were pipetted into each well and incubated for 2 hours at ambient temperature. Following removal of the unbound substances, the biotin-conjugated detecting antibody specific for IFN-γ (Pierce, M701B) was added to the wells and incubated for one hour, respectively. The streptavidin conjugated Horseradish Peroxidase (HRP) (Invitrogen, SNN1004) was then added to the wells for 30 minutes incubation at ambient temperature. The color was developed by dispensing TMB substrate, and then stopped by 2M HCl. The absorbance was read at 450 and 540 nM using a Microplate spectrophotometer.

W3253-U9T2.G17-1.uIgG1V320 could induce human IL-2 (hIL-2) and human IFN-γ (hIFN-γ) secretion in a concentration-dependent manner in mixed lymphocyte reaction assay (FIG. 15A and FIG. 15B).

### 3.11 Human Serum Stability

Antibodies were incubated in freshly isolated human serum (serum content >90%) at 37° C. On indicated time points, an aliquot of serum treated sample were removed from the incubator and snap frozen in liquid N2, and then stored at −80° C. until ready for test. The samples were quickly thawed immediately prior to the stability test. Briefly, plates were pre-coated with 1 μg/mL of W325-hPro1.ECD.his (in house) at 4° C. overnight. After 1-hour blocking, testing antibodies were added to the plates at various concentrations (4-fold serially diluted from 25 nM to 0.0015 nM). The plates were incubated at room temperature for 1 hour. Following the incubation, plates were washed three times with 300 μL per well of PBS containing 0.5% (v/v) Tween 20. 1 μg/ml of W315-hPro1.ECD.mFc (in house) was added to plates and incubation 1 hour. After washing the plates three times, 100 μL 1:5000 diluted HRP-labeled Goat anti mouse IgG (Bethyl A90-231P) was added and incubated on the plates for 1 hour at room temperature. After washing six times with 300 μL per well of PBS containing 0.5% (v/v) Tween 20, 100 μL TMB substrate was added for the detection pre well. The reaction was stopped after approximately 5 minutes through the addition of 100 μL per well of 2 M HCl. The absorbance of the wells is measured at 450 nm and 540 nm with a multiwall plate reader (SpectraMax® M5e).

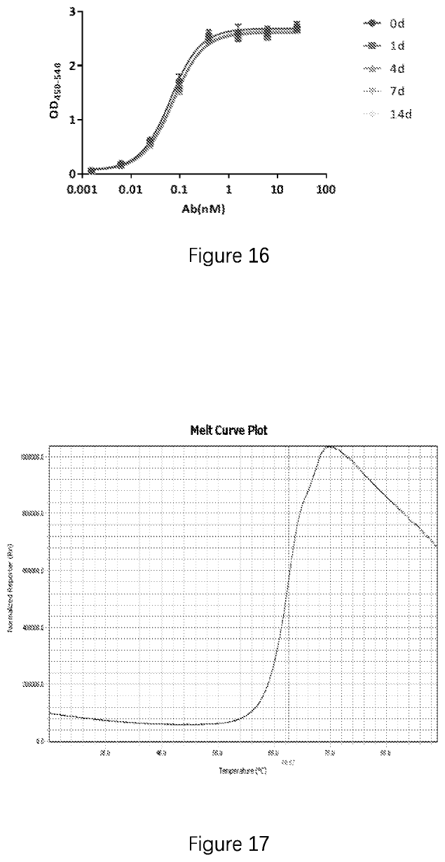

The serum stability of W3253-U9T2.G17-1.uIgG1V320 was assessed under human serum at 37° C. for two weeks. The antibody binding ability treated with human serum for 1 day, 4 days, 7 days and 14 days were comparable with 0 day (FIG. 16).

### 3.12 Thermal Stability (DSF)

Tm of antibodies was investigated using QuantStudio 7 Flex Real-Time PCR system (Applied Biosystems). 19 μL of antibody solution was mixed with 1 μL of 62.5×SYPRO Orange solution (Invitrogen) and transferred to a 96 well plate (Biosystems). The plate was heated from 26° C. to 95° C. at a rate of 0.9° C./min, and the resulting fluorescence data was collected. The negative derivatives of the fluorescence changes with respect to different temperatures were calculated, and the maximal value was defined as melting temperature Tm. If a protein has multiple unfolding transitions, the first two Tm were reported, named as Tm1 and Tm2. Data collection and Tm calculation were conducted automatically by the operation software (QuantStudio□ Real Time PCR software v1.3).

DSF assay results show that the Tm1 value is 68.7° C. (FIG. 17).

### Example 4

### In Vivo Characterization of the Bispecific Antibodies

### 4.1 Mouse PK Study

The pharmacokinetics of WBP3253-U9T2.G17-1.uIgG1V320 was tested in C57BL/6 female mice. Female C57BL/6 mice (Shanghai Lingchang Biotech Co., LTD) of 6-8 weeks-old were used in the study.

For the pharmacokinetics study, three C57BL/6 female mice received an intravenous injection of 13.3 mg/kg of WBP3253-U9T2.G17-1.uIgG1V320. Before antibody injection, pre-dose blood was taken. After antibody injection, in time intervals of 30 min, 2 h, 6 h, 24 h, 2 days, 3 days, 5 days, 7 days and 10 days, blood samples were taken from the eyes and transferred to the EDTA tubes. The tubes were centrifuged at 6000 rpm for 5 min at 4° C., and then the plasma was collected and stored at −20° C.

Serum antibody concentration was determined through three methods by ELISA. Goat Anti-human IgG Fc or W315-hPro1.ECD.mFc, respectively, was immobilized on 96 ELISA plates at 1 μg/ml overnight at 4° C. The plates were washed three times with 100 μl phosphate buffered saline tween (PBST) and the remaining binding sites were blocked with 2% (w/v) Bovine albumin (BSA) at room temperature for 1 h. Purified recombinant antibody, serum sample and QC sample were diluted in 2% BSA, titrated in duplicates and incubated in the ELISA plates at room temperature for 1 h. After washing the plates with PBST for three times, goat Anti-human IgG Fc-biotin (0.0625 μg/ml) or VEGF.his.biotin (1 μg/ml) were incubated at room temperature for 1 h. Detection was performed with HRP-conjugated Streptavidin for using 50 μl tetramethylbenzidine (TMB) substrate. The reaction was stopped with 50 μl of 2M HCl and absorbance was measured at 450-540 nm by SpectraMax®M5e. The standard curve was calculated by the purified recombinant antibodies. Serum antibody concentration was calculated with SoftMax basing on the standard curve. Data were fitted with Graph-Prism software (La Jolla, CA, USA) from 3 independent binding curves. The concentration of serum antibody was analyzed in a non-compartmental model using Phoenix WinNonlin software (version 8.1, Pharsight, Mountain View, CA), parameters of PK were linear log trapezoid rule. The results were represented by mean and the standard deviation (Mean ±SD). The method was summary in PK workflow.

All the procedures related to animal handling, care and the treatment in the study were performed according to the guidelines approved by the Institutional Animal Care and Use Committee (IACUC) of Shanghai WuXibiologics Co., Ltd following the guidance of the Association for Assessment and Accreditation of Laboratory Animal Care (AAALAC).

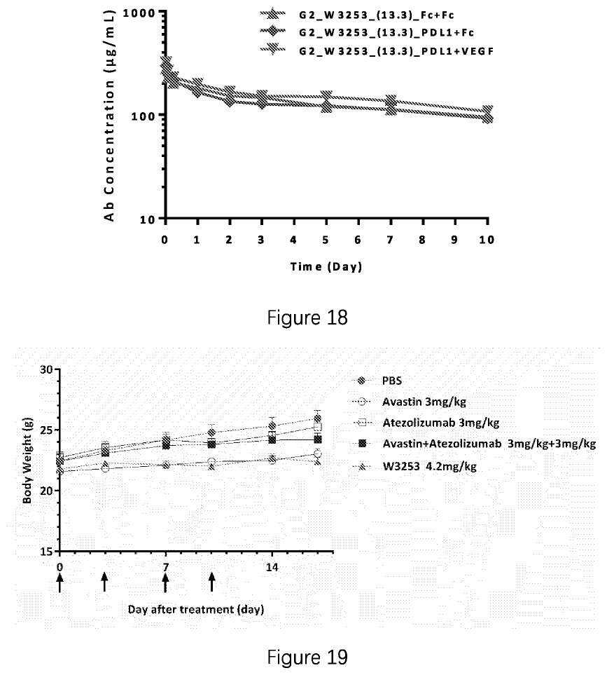

As shown in FIG. 18, the similar results were obtained with the three detection methods, that the antibody was stable in mice. As showed in Table 6, t½ were 292h (Fc+Fc), 342h (Fc+PD-L1), and 356h (PDL1+VEGF), respectively.

TABLE 6
Mouse PK parameters

| | Compound | | |
|---|---|---|---|
| | W3253-U9T2.G17-1.uIgG1V320 | | |
| | Dose | | |
| | 13.3 mg/kg, iv | | |
| Method | Fc + Fc | PD-L1 + Fc | PD-L1 + VEGF |
| t_{1/2} (h) | 292* | 342* | 356* |
| Cₘₐₓ (μg/mL) | 281 | 300 | 321 |
| AUC₀₋ₜ (h*μg/mL) | 31815 | 30479 | 36449 |
| Cl_obs (mL/day/kg) | 4.50 | 4.22 | 3.58 |
| MRTINF_obs (h) | 416 | 484 | 500 |
| Vss_obs (mL/kg) | 75.8 | 83.8 | 70.4 |

*The t_{1/2} may be not accurate when the AUC_% Extrap_obs is greater than 20% or the Rsq_adjusted is less than 0.9.

### 4.2 Exnografted RKO Colonic Carcinoma Tumor Model

WBP3253 in vivo efficacy study was tested in RKO colonic carcinoma tumor model in NCG female mice. Female NCG mice (Nanjing Galaxy Biopharmaceutical Co., LTD) of 8-10 weeks-old were used in the study. RKO cells were maintained in vitro as a monolayer culture in EMEM medium supplemented with 10% fetal bovine serum, 100 U/mL penicillin and 100 μg/mL streptomycin at 37° C. in an atmosphere of 5% CO₂ in air. The tumor cells were routinely sub-cultured twice a week with 0.25% trypsin-EDTA treatment. The cells growing in an exponential growth phase were harvested and counted for tumor inoculation.

For the therapeutic model, each mouse was inoculated subcutaneously at the right front flank with a mix of RKO tumor cells and cryo PBMC (2.0×10⁶ tumor cells and 2.0×10⁶ PBMC mixed with 50% of matrigel in 200 μl PBS). When the average tumor volume reached approximately 137 mm³ after 6 days post inoculation, animals were randomly grouped into 5 groups and each group contained 7 mice. The 5 groups of mice received following intraperitoneal injections respectively: PBS, Avastin (3 mg/kg), Atezolizumab (3 mg/kg), Avastin+Atezolizumab (3 mg/kg+3 mg/kg), W3253-U9T2.G17-1.uIgG1V320 (4.2 mg/kg). The day of intraperitoneal injection was considered as day 0. For all tumor studies, mice were weighed and tumor growth was measured twice a week using calipers. All the procedures related to animal handling, care and the treatment in the study were performed according to the guidelines approved by the Institutional Animal Care and Use Committee (IACUC) of Shanghai SIPPR-BK Laboratory Animal Co., Ltd following the guidance of the Association for Assessment and Accreditation of Laboratory Animal Care (AAALAC).

Tumor volume was calculated with the formula (½ (length×width²). TGI (tumor growth inhalation) was calculated for each group using the formula: TGI (%)=[1−(Ti−T0)/(Vi−V0)]×100. Ti is the average tumor volume of a treatment group on a given day. T0 is the average tumor volume of the treatment group on the first day of treatment. Vi is the average tumor volume of the vehicle control group on the same day with Ti and V0 is the average tumor volume of the vehicle group on the first day of treatment. Relative change of bodyweight change (RCBW) was calculated with the formula [(BWt−BW0)/BW0]×100, BW0 is the average bodyweight at day0, BWt is the average bodyweight at measurement day. The results were represented by mean and the standard error (Mean ±SEM). Data at day 17 were analyzed using Ordinary two-way ANOVA Tukey's multiple comparisons test with Graphpad Prism 6.0 and p<0.05 was considered to be statistically significant.

As shown in FIG. 19, no obvious bodyweight loss was observed on all other animals in each group, which indicated that animals were tolerated well to each test articles.

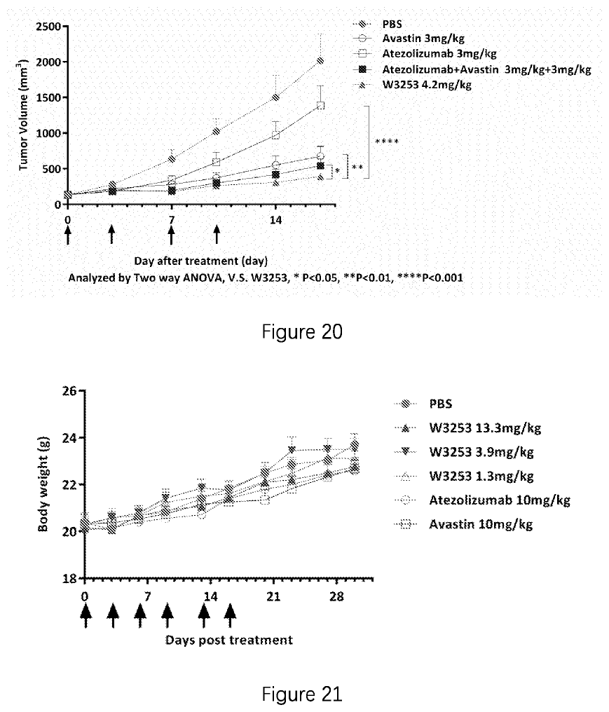

As shown in FIG. 20, at the day17, the average tumor volume of the vehicle control group was 2017 mm³, which indicated RKO xenograft colonic carcinoma tumor model was well established. The TGI at day17 of each group was 69.37% for Avastin, 33.42% for Atezolizumab, 78.37% for Avastin+Atezolizumab combination, 86.55% for W3253-U9T2.G17-1.uIgG1V320. Compared with vehicle control group, all test articles showed inhibition to the tumor growth; Compared to the two monoclonal antibodies, Avastin+Atezolizumab combination and W3253 bispecific antibody showed more potent anti-tumor effect (p<0.001); Compared to the combination, W3253 bispecific antibody showed more potent anti-tumor effect (p<0.05).

### 4.3 Human PD-L1 Knockin MC38 in Human PD1/PD-L1 Dual Knock-In Transgenic Mouse Model

WBP3253 in vivo efficacy study was tested in MC38/hPD-L1 in Human PD1/PD-L1 Dual Knock-in transgenic mouse model in C57BL/6-hPD1/hPDL1 female mice. Female C57BL/6-hPD1/hPD-L1 mice (Nanjing Galaxy Biopharmaceutical Co., LTD) of 8-9 week-old were used in the study. MC38/hPD-L1 cells were maintained in vitro as a monolayer culture in DMEM medium supplemented with 10% fetal bovine serum, 100 U/mL penicillin and 100 μg/mL streptomycin at 37° C. in an atmosphere of 5% CO₂ in air. The tumor cells were routinely sub-cultured twice a week with 0.25% trypsin-EDTA treatment. The cells growing in an exponential growth phase were harvested and counted for tumor inoculation.

For the therapeutic model, each mouse was inoculated subcutaneously at the right front flank with MC38/hPD-L1 tumor cells (1.0×10⁶ tumor cells in 100 ul PBS). When the average tumor volume reached approximately 86 mm³ after 6 days post inoculation, animals were randomly grouped into 6 groups and each group contained 8 mice. The 6 groups of mice received following intraperitoneal injections respectively: PBS, W3253-U9T2.G17-1.uIgG1V320 (13.3 mg/kg), W3253-U9T2.G17-1.uIgG1V320 (3.9 mg/kg), W3253-U9T2.G17-1.uIgG1V320 (1.3 mg/kg), Atezolizumab (10 mg/kg), Avastin (10 mg/kg). The day of intraperitoneal injection was considered as day0. For all tumor studies, mice were weighed and tumor growth was measured twice a week using calipers. All the procedures related to animal handling, care and the treatment in the study were performed according to the guidelines approved by the Institutional Animal Care and Use Committee (IACUC) of Shanghai SIPPR-BK Laboratory Animal Co., Ltd following the guidance of the Association for Assessment and Accreditation of Laboratory Animal Care (AAALAC). Tumor volume was calculated with the formula (½ (length×width²).

TGI (tumor growth inhalation) and RCBW (Relative change of bodyweight change) were calculated as described above. The results were represented by mean and the standard error (Mean ±SEM). Data at day30 were analyzed using Ordinary two-way ANOVA Tukey's multiple comparisons test with Graphpad Prism 6.0 and p<0.05 was considered to be statistically significant.

As shown in FIG. 21, no obvious bodyweight loss was observed on all other animals in each groups, which indicated that animals were tolerated well to each test articles.

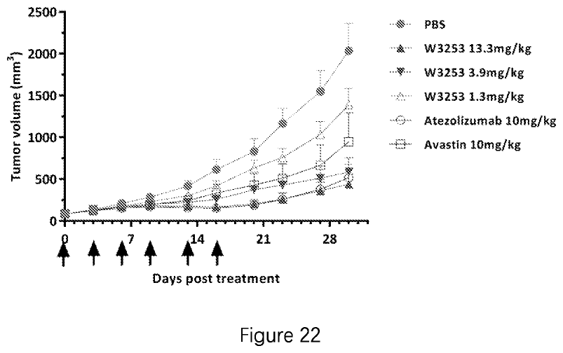

As shown in FIG. 22, at day30, the average tumor volume of the vehicle control group was 2037 mm³, which indicated MC38/hPD-L1 in Human PD1/PD-L1 Dual knock-in transgenic mouse model was well established. The TGI at day 30 of each group was 78.22% for W3253-U9T2.G17-1.uIgG1V320 (13.3 mg/kg), 71.06% for W3253-U9T2.G17-1.uIgG1V320 (3.9 mg/kg), 30.95% for W3253-U9T2.G17-1.uIgG1V320 (1.3 mg/kg), 73.76% for Atezolizumab (10 mg/kg), 52.95% for Avastin (10 mg/kg). Compared with the vehicle control group, all test articles showed inhibition to tumor growth; W3253-U9T2.G17-1.uIgG1V320 bispecific antibody shows comparable tumor inhibition to Atezolizumab at equivalent molar dose level; W3253-U9T2.G17-1.uIgG1V320 showed dose-dependent tumor inhibition.

Those skilled in the art will further appreciate that the present disclosure may be embodied in other specific forms without departing from the spirit or central attributes thereof. In that the foregoing description of the present disclosure discloses only exemplary embodiments thereof, it is to be understood that other variations are contemplated as being within the scope of the present disclosure. Accordingly, the present disclosure is not limited to the particular embodiments that have been described in detail herein. Rather, reference should be made to the appended claims as indicative of the scope and content of the disclosure.

### REFERENCES

[1] Apte R S, Chen D S, Ferrara N. VEGF in Signaling and Disease: Beyond Discovery and Development. Cell. 2019 Mar. 7; 176(6):1248-1264.
[2] Alsaab H O, Sau S, Alzhrani R, et al. PD-1 and PD-L1 Checkpoint Signaling Inhibition for Cancer Immunotherapy: Mechanism, Combinations, and Clinical Outcome. Frontiers in Pharmacology 2017; 8: 561.
[3] Gong, Jun, Chehrazi-Raffle, Alexander et al. Development of PD-1 and PD-L1 inhibitors as a form of cancer immunotherapy: a comprehensive review of registration trials and future considerations. Journal for Immunotherapy of Cancer 2018; 6: 8.
[4] Kudo M. Scientific Rationale for Combined Immunotherapy with PD-1/PD-L1 Antibodies and VEGF Inhibitors in Advanced Hepatocellular Carcinoma. Cancers (Basel). 2020 Apr. 27; 12(5):1089.
[5] Chen D S, Hurwitz H. Combinations of Bevacizumab With Cancer Immunotherapy. Cancer J. 2018 Jul./Aug.; 24(4):193-204.

1-26. (canceled)

27. A bispecific antibody or antigen-binding portion thereof, comprising a PD-L1 antigen-binding moiety associated with a VEGF antigen-binding moiety, wherein:
the PD-L1 antigen-binding moiety comprises:
a heavy chain complementarity determining region (HCDR) 1 comprising the amino acid sequence of SEQ ID NO: 1, a HCDR2 comprising the amino acid sequence of SEQ ID NO: 2, a HCDR3 comprising the amino acid sequence of SEQ ID NO: 3, a light chain complementarity determining region (LCDR) 1 comprising the amino acid sequence of SEQ ID NO: 4, a LCDR2 comprising the amino acid sequence of SEQ ID NO: 5, and a LCDR3 comprising the amino acid sequence of SEQ ID NO: 6; and
the VEGF antigen-binding moiety comprises:
a HCDR1 comprising the amino acid sequence of SEQ ID NO: 7, a HCDR2 comprising the amino acid sequence of SEQ ID NO: 8, a HCDR3 comprising the amino acid sequence of SEQ ID NO: 9, a LCDR1 comprising the amino acid sequence of SEQ ID NO: 10, a LCDR2 comprising the amino acid sequence of SEQ ID NO: 11, and a LCDR3 comprising the amino acid sequence of SEQ ID NO: 12.

28. The bispecific antibody or antigen-binding portion thereof of claim 27, wherein the PD-L1 antigen-binding moiety is a scFv and the VEGF antigen-binding moiety is a Fab.

29. The bispecific antibody or antigen-binding portion thereof of claim 27, wherein:
the PD-L1 antigen-binding moiety comprises a heavy chain variable domain comprising the amino acid sequence of SEQ ID NO: 13 or an amino acid sequence with at least 85%, 90%, or 95% identity to SEQ ID NO: 13 and a light chain variable domain comprising the amino acid sequence of SEQ ID NO:14 or an amino acid sequence with at least 85%, 90%, or 95% identity to SEQ ID NO: 14; and/or
the VEGF antigen-binding moiety comprises a heavy chain variable domain comprising the amino acid sequence of SEQ ID NO: 15 or an amino acid sequence with at least 85%, 90%, or 95% identity to SEQ ID NO: 15 and a light chain variable domain comprising the amino acid sequence of SEQ ID NO:16 or an amino acid sequence with at least 85%, 90%, or 95% identity to SEQ ID NO: 16.

30. The bispecific antibody or antigen-binding portion thereof of claim 27, wherein the PD-L1 antigen-binding moiety is fused to the N terminal of the VEGF antigen-binding moiety.

31. The bispecific antibody or antigen-binding portion thereof of claim 30, wherein the PD-L1 antigen-binding moiety is operably linked to the N terminal of the heavy chain of the VEGF antigen-binding moiety via a linker.

32. The bispecific antibody or antigen-binding portion thereof of claim 31, wherein the linker is a peptide linker comprising or consisting of 1 to 4 copies of GGGGS (G4S).

33. The bispecific antibody or antigen-binding portion thereof of claim 27, comprising a heavy chain and a light chain, wherein:
the heavy chain comprises, from N-terminal to C-terminal, domains operably linked as in scFv-VH-CH1-hinge-Fc, wherein the scFv is from the PD-L1 antigen-binding moiety and the VH-CH1 is from the VEGF antigen-binding moiety; and
the light chain comprises, from N-terminal to C-terminal, domains operably linked as in VL-CL, wherein the VL-CL is from the VEGF antigen-binding moiety.

34. The bispecific antibody or antigen-binding portion thereof of claim 33, wherein the Fc region is a human IgG Fc region.

35. The bispecific antibody or antigen-binding portion thereof of claim 34, wherein the Fc region is a human IgG1 Fc that comprises L234A and L235A substitutions, according to EU numbering.

36. The bispecific antibody or antigen-binding portion thereof of claim 33, wherein the heavy chain comprises the amino acid sequence of SEQ ID NO: 17, and the light chain comprises the amino acid sequence of SEQ ID NO: 18.

37. An isolated nucleic acid molecule, comprising a nucleic acid sequence encoding the bispecific antibody or the antigen-binding portion thereof of claim 27.

38. A vector comprising the nucleic acid molecule of claim 37.

39. A host cell comprising the vector of claim 38.

40. A pharmaceutical composition comprising the bispecific antibody or antigen-binding portion thereof of claim 27 and a pharmaceutically acceptable carrier.

41. A method for producing the bispecific antibody or antigen-binding portion thereof of claim 27, comprising the steps of:
culturing a host cell comprising a nucleic acid sequence encoding the bispecific antibody or antigen-binding portion thereof under a suitable condition; and
isolating the bispecific antibody or antigen-binding portion thereof from the host cell.

42. A method for modulating an immune response in a subject, comprising administering to the subject the bispecific antibody or the antigen-binding portion thereof as defined in claim 27 to the subject, the immune response is PD-L1 and/or VEGF related.

43. A method for preventing or treating cancer in a subject, comprising administering an effective amount of the bispecific antibody or the antigen-binding portion thereof as defined in claim 27 to the subject, wherein the cancer is PD-L1 and/or VEGF related.

44. The method of claim 43, wherein the cancer is colon cancer or colorectal cancer.

45. The method of claim 43, wherein the bispecific antibody or antigen-binding portion thereof is administered in combination with a chemotherapeutic agent, radiation and/or other agents for use in cancer immunotherapy.

46. A kit comprising the bispecific antibody or antigen-binding portion thereof of claim 27.


SEQUENCE LISTING (20 sequences)

SEQ ID NO: 1 - protein, 10 aa, Artificial sequence
  HCDR1
GFTFSSYAMS

SEQ ID NO: 2 - protein, 17 aa, Artificial sequence
  HCDR2
GISGSGGFTYYADSVKG

SEQ ID NO: 3 - protein, 12 aa, Artificial sequence
  HCDR3
PPRGYNYGPFDY

SEQ ID NO: 4 - protein, 11 aa, Artificial sequence
  LCDR1
GGNNIGSKSVH

SEQ ID NO: 5 - protein, 7 aa, Artificial sequence
  LCDR2
DDSDRPS

SEQ ID NO: 6 - protein, 11 aa, Artificial sequence
  LCDR3
QVWDSSSDHVV

SEQ ID NO: 7 - protein, 10 aa, Artificial sequence
  HCDR1
GYTFTNYGMN

SEQ ID NO: 8 - protein, 17 aa, Artificial sequence
  HCDR2
WINTYTGEPTYAADFKR

SEQ ID NO: 9 - protein, 14 aa, Artificial sequence
  HCDR3
YPHYYGSSHWYFDV

SEQ ID NO: 10 - protein, 11 aa, Artificial sequence
  LCDR1
SASQDISNYLN

SEQ ID NO: 11 - protein, 8 aa, Artificial sequence
  LCDR2
YFTSSLHS

SEQ ID NO: 12 - protein, 9 aa, Artificial sequence
  LCDR3
QQYSTVPWT

SEQ ID NO: 13 - protein, 121 aa, Artificial sequence
  VH PDL1
EVQLLESGGGLVQPGGSLRLSCAASGFTFSSYAMSWVRQAPGKGLEWVSGISGSGGFTYYADSVKGRFTISRDNSKNTLYLQMNSLRAEDTAVYYCAKPPRGYNYGPFDYWGQGTLVTVSS

SEQ ID NO: 14 - protein, 108 aa, Artificial sequence
  VL PDL1
SYVLTQPPSVSVAPGQTARITCGGNNIGSKSVHWYQQKPGQAPVLVVYDDSDRPSGIPERFSGSNSGNTATLTISRVEAGDEADYYCQVWDSSSDHVVFGGGTKLTVL

SEQ ID NO: 15 - protein, 123 aa, Artificial sequence [sequence omitted]

SEQ ID NO: 16 - protein, 107 aa, Artificial sequence [sequence omitted]

SEQ ID NO: 17 - protein, 711 aa, Artificial sequence
  Heavy chain
EVQLLESGGGLVQPGGSLRLSCAASGFTFSSYAMSWVRQAPGKGLEWVSGISGSGGFTYYADSVKGRFTISRDNSKNTLYLQMNSLRAEDTAVYYCAKPPRGYNYGPFDYWGQGTLVTVSSGGGGSGGGGSGGGGSGGGGSSYVLTQPPSVSVAPGQTARITCGGNNIGSKSVHWYQQKPGQAPVLVVYDDSDRPSGIPERFSGSNSGNTATLTISRVEAGDEADYYCQVWDSSSDHVVFGGGTKLTVLGGGGSGGGGSEVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 18 - protein, 214 aa, Artificial sequence
  Light chain
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - DNA, 2133 bp, Artificial sequence
  Nucleotide sequence of Heavy chain
gaggtgcaactgttggagtctgggggaggcttggtacagcctggggggtccctgagactctcctgtgcagcctctggattcacctttagcagctatgccatgagttgggtccgccaggctccagggaaggggctggagtgggtctcaggtattagtggtagtggtggtttcacttactacgcagactccgtgaagggccggttcaccatctccagagacaattccaagaacacgctgtatctgcaaatgaacagcctgagagccgaggacacggccgtatattactgtgcgaaacctcctcgtggatacaactatggcccttttgactactggggccagggaaccctggtcaccgtctcctcaggcggcggaggaagcggaggaggtggcagcggtggcggtggctccggcggaggtggctcctcctatgtgctgactcagccaccctcggtgtcagtggccccaggacagacggccaggattacctgtgggggaaacaacattggaagtaaaagtgtacactggtaccagcagaagccaggccaggcccctgtgctggtcgtctatgatgatagcgaccggccctcagggatccctgagcgattctctggctccaactctgggaacacggccaccctgaccatcagcagggtcgaagccggggatgaggccgactattactgtcaggtgtgggatagtagtagtgatcacgtggtattcggcggagggaccaagctgaccgtcctaggtggaggtggcagtggaggcggaggatctgaagttcagctggtggagtctggcggtggcctggtgcagccagggggctcactccgtttgtcctgtgcagcttctggctataccttcaccaactatggtatgaactgggtccgtcaggccccgggtaagggcctggaatgggttggatggattaacacctataccggtgaaccgacctatgctgcggatttcaaacgtcgtttcactttcagcttagacacctccaagtcgacagcatacctgcagatgaacagcctgcgtgctgaggacactgccgtctattactgtgcaaagtacccccactattatgggagcagccactggtatttcgacgtctggggtcaaggaaccctggtcaccgtctcctcggcgtcgaccaagggcccatccgtcttccccctggcaccctcctccaagagcacctctgggggcacagcggccctgggctgcctggtcaaggactacttccccgaaccggtgacggtgtcctggaactcaggcgctctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgccgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtctacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcttaagcctgtctccgggt

SEQ ID NO: 20 - DNA, 642 bp, Artificial sequence
  Nucleotide sequence of Light chain
gatatccagatgacccagtccccgagctccctgtccgcctctgtgggcgatagggtcaccatcacctgcagcgcaagtcaggatattagcaactatttaaactggtatcaacagaaaccaggaaaagctccgaaagtgctgatttacttcacctcctctctccactctggagtcccttctcgcttctctggatccggttctgggacggattttactctgaccatcagcagtctgcagccagaagacttcgcaacttattactgtcaacagtatagcaccgtgccgtggacgtttggacagggtaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt